(19) United States
(12) Patent Application Publication
(10) Pub. No.: US 2011/0263953 A1
(43) Pub. Date: Oct. 27, 2011

(54) DEPLOYMENT SYSTEM AND METHOD FOR OPTICAL ANALYTE SENSOR

(75) Inventors: David R. Markle, Berwyn, PA (US); Soya Gamsey, Huntington Beach, CA (US); Kenneth J. Chesnin, Irvine, CA (US)

(73) Assignee: GLUMETRICS, INC., Irvine, CA (US)

(21) Appl. No.: 13/095,748

(22) Filed: Apr. 27, 2011

Related U.S. Application Data
(60) Provisional application No. 61/328,590, filed on Apr. 27, 2010.

(51) Int. Cl. A61B 5/1455

(52) U.S. Cl. 600/310

(57) ABSTRACT

Embodiments of the invention are directed to a delivery device and method for deploying an optical analyte sensor. The delivery device comprises hollow tubes configured to operate telescopically. The optical sensor is configured to retract and extend from a distal end of the delivery device, by sliding the telescoping tubes with respect to one another. The delivery device may also have a locking mechanism such that the distal end portion of the sensor will extend to a preset locked position beyond the delivery device, e.g., during calibration and deployment. The device is capable of being used to ship, calibrate, and deploy the sensor while maintaining sterility and avoiding exposure to the external environment.

### CROSS-REFERENCE TO RELATED APPLICATIONS

This application claims priority to U.S. Provisional Patent Application No. 61/328,590, filed Apr. 27, 2010 the disclosure of which is hereby expressly incorporated by reference and hereby expressly made a portion of this application.

### BACKGROUND OF THE INVENTION

1. Field of the Invention

Embodiments of the invention are directed to a deployment system comprising an optical sensor for detecting an analyte, preferably glucose, and a telescoping deployment device.

2. Description of the Related Art

Improved glycemic control requires continuous and accurate monitoring of a patient's blood glucose level. Thus, there is a need for a real-time glucose monitoring system that is adapted to meet the needs of ICU patients. Furthermore, there continues to be a need for a method of effectively preparing, calibrating, and deploying the analyte sensor assembly without compromising the sensor chemical integrity and sterility. In certain existing monitoring systems, the sensor is calibrated using a calibration solution that often requires the sensor to be exposed to the environment, which may adversely affect the sensor chemistry or sterility.

### SUMMARY OF THE INVENTION

An analyte detection system is disclosed. The system comprises an optical fiber sensor comprising proximal and distal end portions and a chemical indicator system disposed along the distal end portion, the chemical indicator system comprising a fluorophore and an analyte binding moiety, which interact to generate a fluorescent signal related to an amount of analyte bound to the analyte binding moiety, and a deployment device comprising telescoping tubes configured to extend and retract at least the distal end portion of the optical fiber sensor, such that sensor integrity, hydration and sterility is maintained during sensor calibration and deployment. The deployment device can further comprise a latch, catch, snap-together coupling, or any locking mechanism that can stop sliding of the telescoping tubes with respect to one another, at a preset fixed or a selected adjustable position, such that the sensor is extended by a desired length. In one embodiment, the latch or catch may be located on the telescoping tubes and can engage with a base, also located on the telescoping tubes, such that when the latch is engaged with the base, the distal end portion of the optical fiber sensor extends by a predetermined length. The deployment device can further comprise a connector at the proximal end of the deployment device that can both electrically and optically connect with a cable extending from the monitor device, wherein the connector can transmit electrical signals and optical signals to and from the monitor device. The deployment device can further have a seal and/or gasket installed so that there is a seal maintained between the telescoping tubes and the optical fiber passing concentrically therein.

Also disclosed are deployment devices for delivering the sensor into a patient, e.g., a blood vessel or interstitial space. The deployment device may comprise an optical fiber as disclosed above with a chemical indicator system positioned along the distal end of the optical fiber. The delivery device may comprise a first hollow tube, a second extension tube configured to extend from the first hollow or retract into the first hollow tube. A third hollow tube with a distal connector end has a proximal end that is connected to the distal end of the second tube. The distal connector can connect to various external devices, such as a cannula, a calibration chamber, testing module, or a storage chamber. The deployment device can further comprise a multi connector junction, such as a Y junction, that can connect to a second external device, such as a syringe with sterile solution to flush the delivery port area.

The deployment device is capable of delivering the analyte sensor to various external devices through the distal connector without compromising the sterility or damaging the chemical sensor at the tip of the fiber optic. For example, by connecting the distal connector of the deployment device to a calibration device, then extending the sensor into the calibration device and calibrating, followed by retracting the analyte sensor into the deployment device, the user is capable of calibrating the sensor without exposing the sensor. After calibration, the user can connect the distal connector to a cannula and extend the sensor into the patient in a similar protected manner. Also disclosed is a method of calibrating the sensor inside the deployment device, without the use of an external calibration device. A calibration buffer injector is connected to the distal end of the deployment device and then calibration buffer is injected into the deployment device, wherein the distal end portion of the optical fiber sensor is submerged into the calibration buffer. The calibration buffer can be heated, e.g., to physiologic temperature (37° C.), by various mechanisms, such as heating tape or heating chamber. After calibrating the sensor, the calibration buffer is removed.

### BRIEF DESCRIPTION OF THE DRAWINGS

FIG. 1A is a schematic side view of a prior art device.

FIG. 1B is a schematic side view of a deployment device of the present invention.

FIG. 2A shows a perspective view of a latch and base mechanism of the deployment device in the engaged position.

FIG. 2B shows a perspective view of a latch and base mechanism of the deployment device in the disengaged position

FIG. 3A is a perspective view of schematic side view of a deployment device of the present invention.

FIG. 3B shows a cross section view of the latch and base mechanism of the deployment device in the engaged position.

FIG. 4A shows a perspective view of a deployment device with a removable lock and key mechanism, wherein the device is in the extended state and the lock removed.

FIG. 4B shows a perspective view of the deployment device with a removable lock and key mechanism, wherein the device is in the retracted state and the lock attached.

FIG. 5A shows a perspective view of a deployment device with a toggle lock, wherein the device is in the extended state and the lock is open.

FIG. 5B shows a perspective view of the deployment device with a toggle lock, wherein the device is in the retracted state and the lock is closed.

FIG. 6A shows a perspective view of a deployment device with a snap-in lock.

FIG. 6B shows a side view of the deployment device with a snap-in lock.

FIG. 7A shows a cross-section of an adjustment module attached to the deployment device.

FIG. 7B shows a cross-section view of the adjustment module attached to the deployment device.

FIG. 8A shows a cross-section view of an adjustment module attached to the deployment device.

FIG. 8B shows a perspective view of the adjustment module attached to the deployment device.

FIG. 9A shows a perspective view of an adjustment module attached to the deployment device.

FIG. 9B shows a schematic view of the adjustment module attached to the deployment device.

FIG. 10A shows a schematic view of a sensor connector.

FIG. 10B shows a front view of the sensor connector.

FIG. 10C shows a side view of the sensor connector.

FIG. 11A shows a schematic view of a monitor cable.

FIG. 11B shows a close up view of the optical ferrule element of the monitor cable.

FIG. 12A shows a schematic view of the monitor cable connector.

FIG. 12B shows a front view of the monitor cable connector.

FIG. 13A shows a schematic view of a distribution of various optical fibers of the monitor cable.

FIG. 13B shows a schematic view of another distribution of various optical fibers of the monitor cable.

FIG. 14A shows a cross-sectional view of one embodiment of a glucose sensor having a cavity in the distal portion of the sensor and a temperature probe.

FIG. 14B shows a perspective view of the glucose sensor.

FIG. 15 shows a cross-sectional view of another embodiment of a glucose sensor having a cavity in the distal portion of the sensor.

FIG. 16 shows a cross-sectional view of another embodiment of a glucose sensor having a cavity in a distal portion of the sensor enclosed within a cage and an additional reference material.

FIG. 17 shows a cross-sectional view of another embodiment of a glucose sensor having a cavity in a distal portion of the sensor and an additional reference material.

FIG. 18 shows a schematic view of another embodiment of a glucose sensor having a glucose sensing optical fiber adjacent to a reference optical fiber.

FIG. 19 shows a glucose measurement system comprising one excitation light source, a single exciter-dual emitter fluorophore system, and a microspectrometer and/or spectrometer.

FIG. 20A through FIG. 22B are perspective views and cross section views of the gasket of the deployment device.

### DETAILED DESCRIPTION OF THE PREFERRED EMBODIMENT

Various embodiments of optical systems and methods are disclosed herein for determining blood glucose concentrations. In certain embodiments, the preferred glucose sensors described herein measure glucose “activity” as opposed to glucose concentration. More precisely, glucose activity refers to the amount of free glucose per kilogram of water. In some embodiments, glucose activity can be measured directly using glucose sensors, such as the equilibrium, non-consuming optical glucose measurement systems discussed above, which employ a chemical indicator system to quantify the amount of free, bioavailable glucose, which is in equilibrium between the water compartment of blood (i.e., not associated with cells, proteins or lipids, etc.) and the glucose binding moiety/quencher. The discussion of the sensors that follow will often refer to the physical quantity to be measured as an “analyte concentration”, “glucose concentration” or simply a “concentration.” However, it is to be understood that “concentration” as used herein, refers to both “analyte concentration” as that phrase might ordinarily be used (e.g., mg/dL or mmol/L blood or plasma) and also to “activity” (in some cases “glucose activity”) as that phrase is described above (e.g., mmol/Kg water). Conversion between the more typical “glucose concentration” (mg/dL) and the potentially more physiologically relevant “glucose activity” (mg/Kg water) can be made, with use input of patient hematocrit, as described in co-pending PCT application No. PCT/US2010/061163; incorporated herein in its entirety by reference thereto.

In certain embodiments, the optical systems and methods disclosed herein involve exciting a chemical indicator system with an excitation light signal and measuring the emission light signal of the indicator system, wherein the indicator system is in contact with the blood and comprises a fluorescent dye operably coupled to a glucose binding moiety—such that the emission light signal generated by the indicator system upon excitation is related to the blood glucose concentration.

The embodiments may further involve correcting the blood glucose concentration measurements from the indicator system for potential artifacts due to the optical system, which artifacts are unrelated to the blood glucose concentration. The correction is performed by ratiometric analysis. More particularly, the ratio of emission light signal to a second light signal that is propagated through the optical system, e.g., the excitation light signal or a separate reference light signal, is used for correcting any non-glucose related contributions of the optical system, as detailed in US Appl. No. 2008/0188725. Ratiometric correction of measured glucose activity or concentration for pH variations can also be made as detailed in U.S. Pat. No. 7,751,863.

Various structural configurations have been proposed for holding a chemical indicator system in a position, which is: (1) exposed to the blood, (2) disposed within the excitation light path, and (3) for exposing a chemical indicator system to the blood and, for introducing to the indicator system an excitation light signal, for detecting an emission light signal from the indicator system, and for enabling ratiometric correction of glucose determinations for artifacts of the system optics; see in particular 2008/0188725.

Optical glucose sensors, such as those described in U.S. Pat. No. 7,417,164, 7,751,863, 7,767,846, 7,824,918, 7,829,341 and U.S. Patent Publ. Nos. 2008/0188725, 2008/0187655, 2008/0305009, 2009/0018426, 2009/0018418, 2009/0264719, 2010/0312483, 2011/0077477 and co-pending U.S. patent application Ser. Nos. 11/296,898, 12/274,617, 12/972,385 and 13/046,571, 13/022,494, 61/378,728 and PCT Appl. Nos. PCT/US2010/061163, PCT/US2010/061173, PCT/US2011/023939 (each of which is incorporated herein in its entirety by reference thereto) typically employ a chemical indicator system disposed at the distal end of an optical fiber, wherein the indicator system is maintained in contact with the blood, such that an excitation light signal sent distally down the fiber causes the chemical indicator system to emit a light signal related to the concentration of glucose.

In certain embodiments, an optical glucose measurement system is disclosed for measuring glucose concentration in blood using one or more glucose-sensing chemical indicator systems. Such indicator systems preferably comprise a fluorophore operably coupled to a glucose binding moiety. Preferably, the glucose binding moiety acts as a quencher with respect to the fluorophore (e.g., suppresses the fluorescent emission signal of the fluorophore in response to excitation light when it associates with the fluorophore).

In certain embodiments, the optical glucose measurement system measures glucose concentrations intravascularly or interstitially, and in real-time through the use of such chemical indicator systems. In certain embodiments, the glucose-sensing chemical indicator systems are immobilized in a hydrogel. The hydrogel may be inserted into an optical fiber such that light may be transmitted through the hydrogel while at least a portion of the hydrogel is in contact with blood. The hydrogel is preferably permeable to blood and analytes, specifically glucose. In certain embodiments, the optical fiber together with the hydrogel comprises a glucose sensor that is placed in a mammalian (human or animal) blood vessel.

Examples of glucose-sensing chemical indicator systems and glucose sensor configurations for intravascular glucose monitoring include the optical sensors disclosed in U.S. Pat. Nos. 5,137,033, 5,512,246, 5,503,770, 6,627,177, 7,417,164, 7,470,420 and 7,751,863, and U.S. Patent Publ. Nos. 2008/0188725, 2008/0187655, 2008/0305009, 2009/0018426, 2009/0018418, and co-pending U.S. patent application Ser. Nos. 11/296,898, 12/187,248, 12/172,059, 12/274,617 and 12/424,902; each of which is incorporated herein in its entirety by reference thereto.

Light may be transmitted into an optical glucose sensor from a light source. In certain embodiments, the light source is a light emitting diode that emits an optical excitation signal. The optical excitation signal excites the fluorophore system(s), such that the fluorophores emit light at an emission wavelength. In certain embodiments, the fluorophore systems are configured to emit an optical emission signal at a first wavelength having an intensity related to the blood glucose concentration in the blood vessel. In certain embodiments, light is directed out of the glucose sensor such that the light is detected by at least one detector. The at least one detector preferably measures the intensity of the optical emission signal, which is related to the glucose concentration present in the blood. Various optical configurations for interrogating glucose-sensing chemical indicator systems with one or more excitation light signals and for detecting one or more emission light signals from the chemical indicator systems may be employed, see e.g., U.S. patent application Ser. No. 12/027,158 (published as 2008/0188725); incorporated herein in its entirety by reference thereto.

### In Vivo Deployment

In one embodiment, the present invention involves a method for deploying a sensor in a blood vessel of a patient, wherein the sensor resides within the patient without any additional device or structural components, e.g., introducer, cannula, catheter, sleeve, etc; see e.g. US 2009/0264719 A1 (incorporated herein in its entirety by reference thereto). The deployment of a naked, preferably very small and non-thrombogenic, sensor addresses some of the disadvantages that presently face patients and medical staff, e.g., thrombogenesis, constant staff care, etc. The sensor can be adapted to sense any analyte using known sensing systems and/or chemistries. The blood vessel can be an artery or a vein. The method comprises positioning the sensor in the blood vessel, such that a distal portion of the sensor resides within the blood vessel by itself, and a proximal portion of the sensor extends out of the patient, wherein there are no additional components associated with the sensor within the patient.

More particularly, new and elegant solutions to some of the technical challenges faced by patients and medical staff in using existing in vivo glucose sensors are disclosed. In one embodiment, a solution to the technical challenges involves using a sensor comprising equilibrium, non-consuming fluorescence-based detection chemistry. Equilibrium optical sensing addresses the problems associated with rate-limiting consumption of enzymatic reactants in current electrochemical sensors. Further, placement within a peripheral vein, as opposed to subcutaneous (interstitial) placement, provides direct monitoring of blood glucose levels, thereby avoiding the problems associated with measuring glucose levels in the interstitial fluid—e.g., uncertain and changing equilibration time for glucose between the blood and the interstitial fluid. In another embodiment, deployment of a very small diameter, non-thrombogenic fiber-optic sensor within a vein, without an indwelling cannula, addresses the serious burden on the nursing staff related to continuous or periodic flushing of the cannula to maintain open access to the vein.

In alternative embodiments, it may be desirable to deploy an optical fiber sensor into a vein or artery within an indwelling cannula, which is not removed. Such embodiments may facilitate nursing care, flushing, anchoring, etc.

### Optical Glucose Sensor Configurations

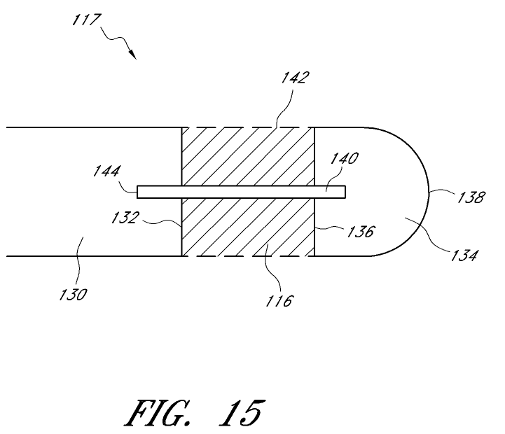

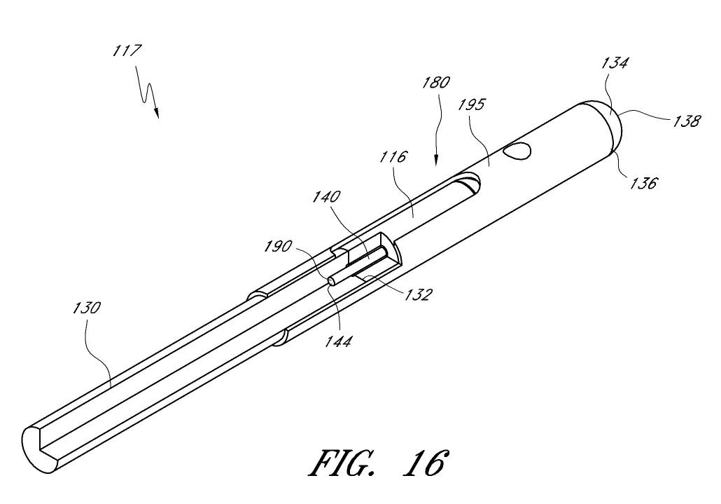

Various glucose sensor configurations are possible as disclosed in U.S. application Ser. No. 12/612,602 filed Nov. 4, 2009, which is incorporated herein in its entirety by reference. In certain embodiments, as illustrated in FIGS. 14(A & B)-15, the glucose sensor 117 comprises an optical fiber 130 having a distal end 132, an atraumatic tip portion 134 having a proximal end 136 and a distal end 138, a void or cavity 116 between the distal end 132 of the optical fiber 130 and the proximal end 136 of the atraumatic tip portion 134, and a rod 140 connecting the distal end 132 of the optical fiber 130 to the proximal end 136 of the atraumatic tip portion 134, wherein the rod traverses the void or cavity. In preferred embodiments, molecules of a chemical indicator system are disposed within the void or cavity 116 and immobilized (by covalent bonding or non-covalent interaction) or otherwise associated within hydrogel matrices. See e.g., the chemical indicator systems disclosed in U.S. Pat. Nos. 7,417,164 and 7,470,420. The cavity 116 may be loaded with hydrogel/chemical indicator system by any methods known in the art. In preferred embodiments, the cavity 116 is filled with hydrogel/chemical indicator system in a liquid state. The hydrogel/chemical indicator systems are preferably polymerized in situ, as detailed in co-pending U.S. patent application Ser. No. 12/026,396 (published as 2008/0187655).

In some embodiments, the proximal surface of the rod 144 is reflective so that a portion of the excitation light signal (or reference light signal) is reflected proximally down the optical fiber 130 to a detector (not shown). The term rod is used herein to refer to any elongate structural member, regardless of its geometry, configured to connect the atraumatic tip portion to the optical fiber

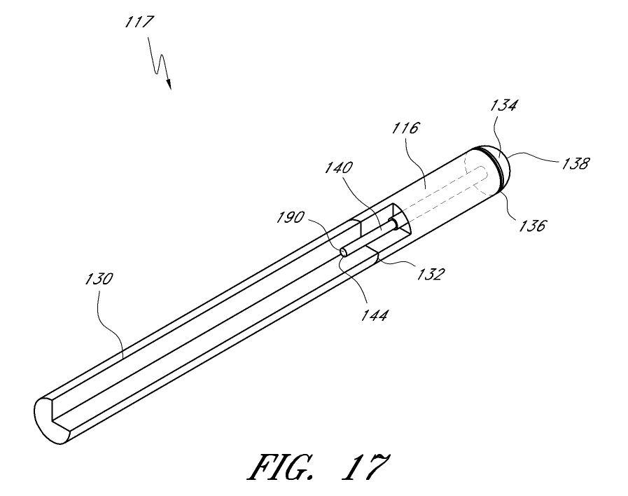

In certain embodiments, as illustrated in FIGS. 16-17, a reference material 190 may be attached to the proximal surface of the rod 144. The reference material 190 may be reflective (e.g., a mirror) and functions similar to embodiments in which the proximal surface of the rod 144 reflects at least a portion of the excitation light signal (or reference light signal) down the optical fiber 130 to a detector (not shown). In other embodiments, the reference material 190 comprises a separate dye indicator system, such as for example a glucose-insensitive fluorescent dye.

The hydrogel and glucose-sensing chemical indicator system is disposed within the cavity 116. In preferred embodiments, the hydrogel/chemical indicator system filled cavity 116 is covered by a selectively permeable membrane 142 that allows passage of glucose into and out of the hydrogel/chemical indicator system. Although these embodiments are described using a glucose sensor 117, it should be understood by a person of ordinary skill in the art that the sensor 117 can be modified to measure other analytes by changing, for example, the sensing chemistry, and if necessary, the selectively permeable membrane 142.

In some embodiments, as illustrated in FIG. 16, the glucose sensor 117 includes a cage 195, as an outer shell, connecting the atraumatic tip 134 with the optical fiber 130. The cage 195 can add mechanical stability to the distal portion of the sensor 117. In some embodiments, the cage 195 also adds flexibility to the distal portion of the sensor 117, allowing the atraumatic tip portion 134 to flex back and forth relative to the orientation of the optical fiber 130. The flexibility of the cage 195, and thus the degree which the atraumatic tip portion 134 can flex, can be increased or decreased by decreasing or increasing the thickness of the cage 195 walls. In addition, the flexibility of the cage 195 can be altered by making the cage 195 from a stiff or flexible material, including e.g., a braided material. In some embodiments, the diameter of the optical fiber 130 may be smaller than the diameter of the interior of the cage 195, allowing the optical fiber 130 to fit within the interior of the cage 195 and abut the void or cavity 116.

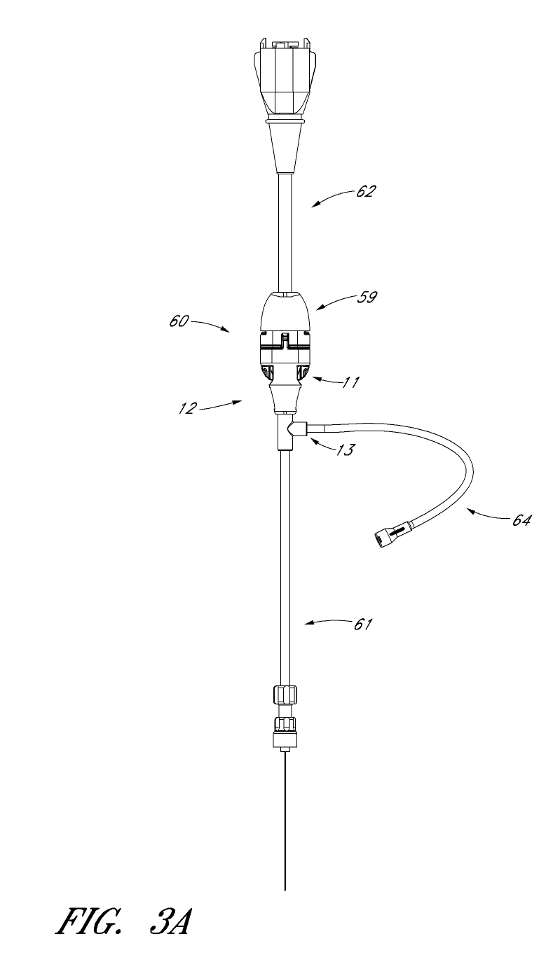

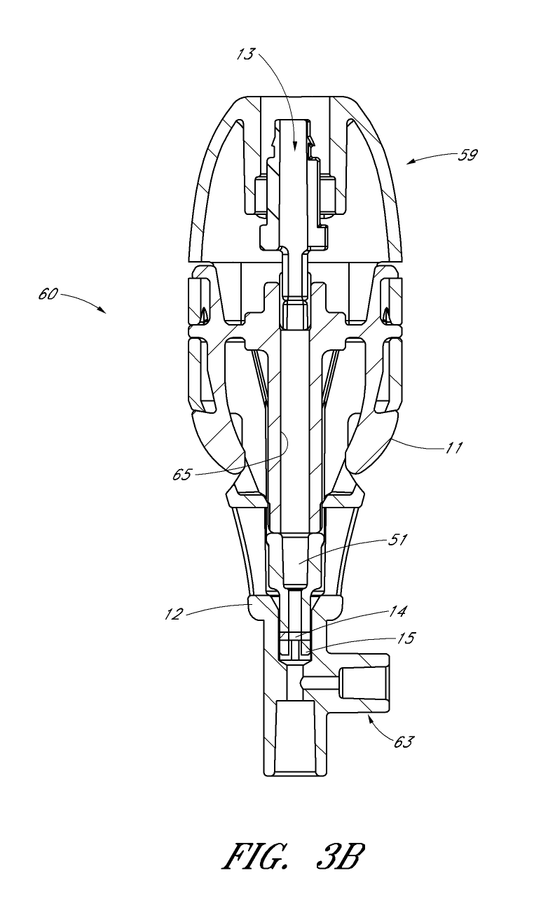

In some embodiments, as illustrated in FIG. 17, the glucose sensor 117 does not have a cage 195 surrounding the void or cavity 116. Instead, similar to FIGS. 14(A & B)-15, the rod 140 connects the optical fiber 130 and atraumatic tip 134, providing structure for the glucose sensor 117, and is surrounded by the void or cavity 116, which in turn is covered by a selectively permeable membrane 142. Similar to FIG. 3, the diameter of the optical fiber 130 is the same as the diameter of the hydrogel/chemical indicator system encased cavity 116. As discussed with respect to FIG. 16, the rod may have a reference material 190 attached to the proximal surface of the rod 144, which functions as previously discussed.

A person skilled in the art would readily understand that the above described embodiments, or components of the above described embodiments, may be combined within the scope of the present invention. For example, a glucose sensor may contain one or more structural elements, such as a cage, a hypotube, and/or a rod within the scope of the present invention. In addition, a glucose sensor may contain one or more reference materials, functioning as a reflective surface and/or as a separate dye indicator system, in different locations and configurations within the scope of the present invention.

In some embodiments, the glucose sensor 117 comprises an atraumatic tip portion 134 (as illustrated in FIGS. 14-17). The atraumatic tip portion 134 has a distal end 138 that is curved and substantially free of sharp edges. In addition, the atraumatic tip portion 134 can be flexible and deformable. The distal end 138 of the atraumatic tip portion 134 can be hemispherical, parabolic, elliptical or curved in any other suitable shape that is reduces the risk of injury to the patient. The atraumatic tip portion 134 can be made from a variety of materials, such as plastics, polymers, gels, metals and composites of the above.

In some embodiments, e.g., as illustrated in FIG. 14A, the glucose sensor 117 includes a temperature sensor or probe 146, such as thermocouple or thermistor. The temperature sensor 146 can measure the temperature of the hydrogel and glucose sensing chemistry system, and/or the blood when disposed intravascularly. The temperature sensor 146 is particularly preferred when the glucose-sensing chemistry is affected by temperature. For example, in some embodiments, the fluorescence intensity emitted by the fluorophore system is dependent on the temperature of the fluorophore system. By measuring the temperature of the fluorophore system, temperature induced variations in fluorophore fluorescence intensity can be accounted for, allowing for more accurate determination of glucose concentration.

In certain embodiments, the temperature sensor can be a thermistor (as described above with regard to FIG. 14A, reference numeral 146, a platinum resistance temperature device (“RTD”), another RTD, a thermocouple, an infrared-based temperature detector, a fluorescence-based temperature sensing element, or other temperature sensing elements with determinable temperature-dependent characteristics.

In another embodiment, a fluorescence-based temperature sensing technique can be used. Fluorescence-based temperature sensing techniques include those based on fluorescence decay, such as where an excitation light is provided to a phosphor, the excitation light is stopped, and the fluorescence is monitored versus time, with the rate of decrease in fluorescence being related to the temperature of the phosphor. Various techniques, can also include phase measurement and phase angle analysis.

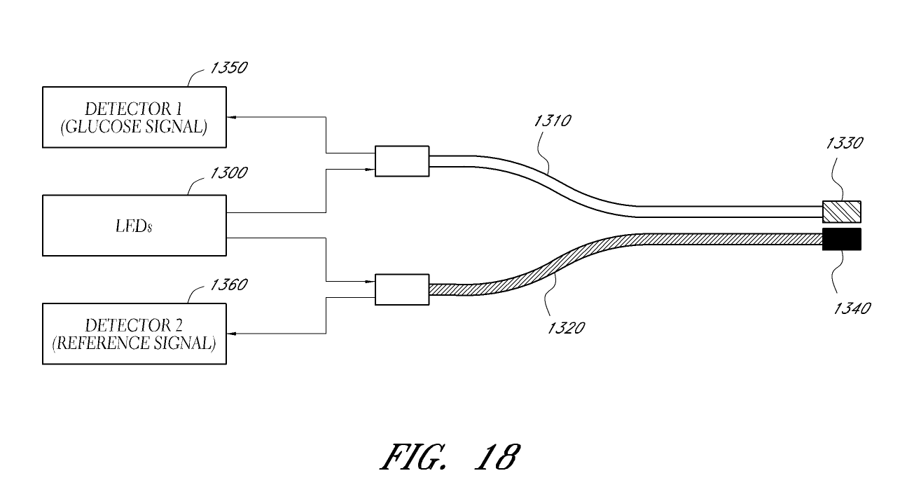

FIG. 18 illustrates another embodiment for measuring the glucose concentration in comparison to a reference signal. In this embodiment, a LED source 1300 sends an excitation signal down two separate adjacent optical fibers 1310, 1320. The first optical fiber, or the glucose fiber 1310, has a proximal tip and a distal tip. The distal tip has a glucose sensing hydrogel 1330 which contains a fluorophore or dye, a quencher, and glucose binding receptors. The second optical fiber, or the reference fiber 1320, also has a proximal tip and a distal tip. The distal tip of the reference fiber has a reference material 1340. In certain embodiments, the reference material 1340 contains the same or a different fluorophore or dye, may or may not contain the quencher, but does not contain glucose receptors. In other embodiments, the reference material 1340 has the same exact glucose sensing hydrogel, but it is encased in a glucose impermeable membrane. In both of these embodiments, the reference fiber 1320 emits a fluorescent return signal independent of the glucose concentration.

After the excitation light passes through the glucose fiber 1310 and the reference fiber 1320, the glucose sensing hydrogel 1330 and the reference material 1340 emit fluorescent signals back to two separate detectors, a glucose signal detector 1350 and a reference signal detector 1360, for ratiometric processing. The benefit of the dual fiber configuration is that both fibers 1310, 1320 experience the same external pressure, bending, temperature, and other external factors. In addition, both fibers 1310, 1320 contain substantially the same material in the glucose sensing hydrogel 1330 and reference material 1340. As a result, the ratio of the intensities between the two fibers 1310, 1320, as measured by the detectors 1350, 1360, produce a calibrated glucose signal that removes, inter alia, the effect of the fluctuations in the LED output or altered transmission along the optical fiber, and thereby increase the accuracy in the measurement of the glucose concentration.

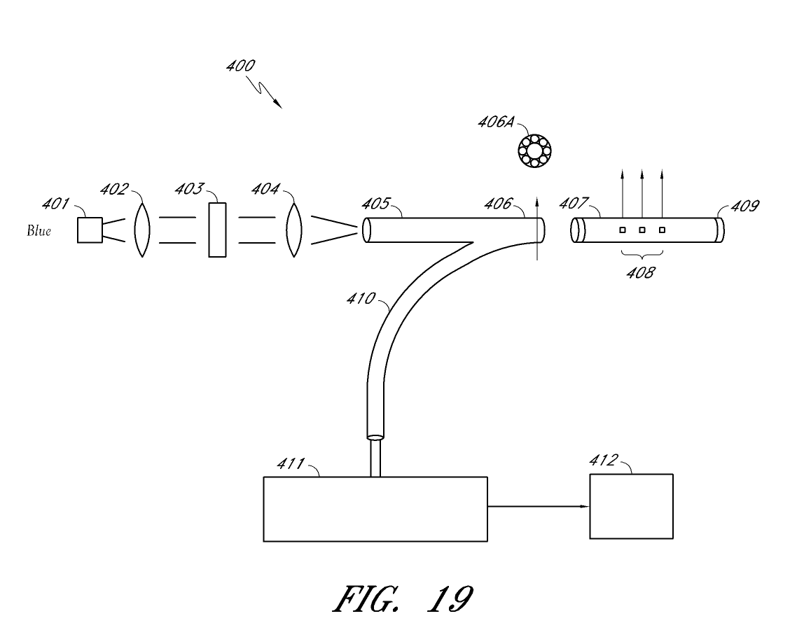

With reference to FIG. 19, in certain embodiments, the light generated by the single light source 401 is transmitted through a optical module comprising a collimator lens 402, an interference filter 403, and/or a focusing lens 404 as described above. The resulting light can be filtered through an interference filter 403. The resulting light can be focused by a focusing lens 404 into an optical fiber 405, which may be a single fiber or a bundle of fibers. The optical fiber 405 can surround optical fiber 410 as both fiber optic lines connect to the first end of the glucose sensor 407. In certain embodiments, a mirror or reflective surface 409 is attached to the second end of the glucose sensor 407. The optical fiber 410 may be a single fiber or a bundle of fibers. The glucose sensor can comprise hydrogels that further comprise a fluorophore system that produces two emission wavelengths, a first emission wavelength and a second emission wavelength. In certain embodiments, the fluorophore system is excited by the light generated by light source 401. In certain embodiments, the optical fiber 410 is connected to a light sensitive module comprising a microspectrometer 411 that measures the entire spectrum of light in the glucose measurement system 400. Data from the microspectrometer 411 can be transmitted to computer 412 for processing. The microspectrometer 411 can allow system 400 to simultaneously measure the excitation light intensity as well as both emission light intensities. In other embodiments, optical fiber 410 may be coupled to other means for quantifying e.g., emission and reference light signals, such as beam splitters, collimating lenses, filters, detectors, amplifiers, etc. as detailed in U.S. Patent Publ. No. 2008/0188725. Ratiometric calculations may be employed to substantially eliminate or reduce non-glucose related factors affecting the intensity of the measured emission light and measured excitation light (also as detailed in U.S. Patent Publ. No. 2008/0188725; incorporated herein in its entirety by reference thereto). The measured emission light can be divided by the measured excitation light, wherein such calculations substantially eliminate or reduce non-glucose related factors affecting the intensity of the lights.

In certain preferred embodiments, the fluorophore dye may be selected such that it exists in distinguishable acid and base conformations, each of which emit at a distinct wavelength, and wherein the relative proportion of acid and base forms depend on the pH. The ratio of intensities of the acid and base emissions can be used to determine the pH of the blood (as detailed in U.S. Pat. No. 7,751,863; incorporated herein in its entirety by reference thereto). The ratio of the acid or base emission intensity over the excitation light can be used to determine the level of glucose in the blood. Of course in a variation to this single exciter-dual emitter fluorophore system, one could employ a single exciter-single emitter for detection of glucose concentration without simultaneous ratiometric determination of pH. Indeed, a great variety of design options are available (see e.g., U.S. Patent Publ. No. 2008/0188725 and U.S. Pat. No. 7,751,863), wherein the chemical indicator and optical systems may be selected based on the preferred use.

### Delivery Device

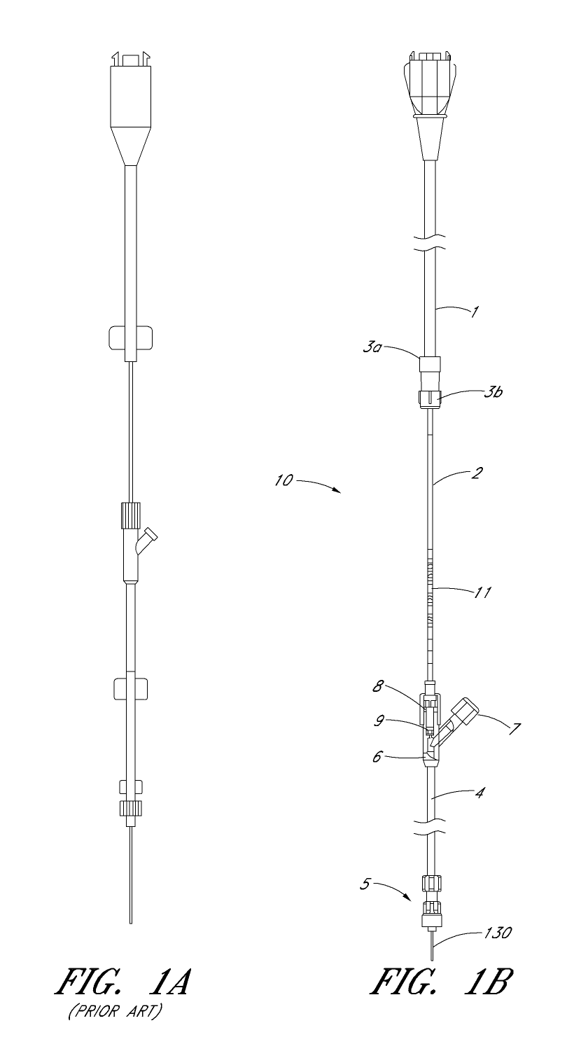

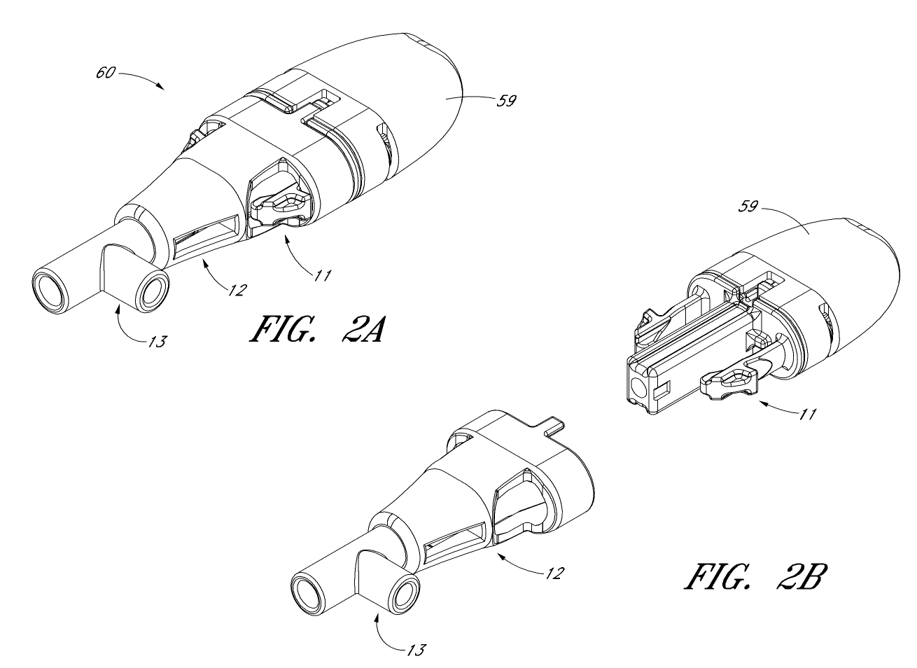

FIG. 1A illustrates a prior art delivery device consisting of a first and second tube being connected telescopically (as detailed in U.S. Pat. No. 5,596,988, which is incorporated herein in its entirety by reference thereto). FIG. 1B illustrates an embodiment of a delivery or deployment device of the present invention. The delivery or deployment system comprises a combination of an optical fiber 130 and a deployment device 10. Of course the optical fiber is just one type of sensor or medical device that can be deployed using the delivery or deployment device described herein. In certain embodiments, the deployment device has a telescoping tube element made of a first tube 1 having a distal end and a proximal end, and a second tube 2 configured to extend and/or retract. The second tube 2 has an outer diameter the same as or slightly less than the inner diameter of the first tube 1, and the second tube 2 is concentrically disposed within the distal portion of the first tube 1 so that it may be telescopically extended beyond the distal end of the first tube 1 or retracted within the distal portion of the first tube 1. In certain embodiment, the telescoping tubes comprise interacting multiple hollow tubes with several subparts to add flexibility on the amount of retractions and extensions it can carry out.

The second tube 2 can be a collapsible material, such as a soft and/or thin polymer that is easily compressible, which allows the second tube 2 to retract into the first tube 1 even though the proximal end of the second tube 2 is abutting the proximal end of the internal tube in its fully retracted state. In another embodiment, the second tube 2 crumples or compresses without creating substantial resistance of force. In one embodiment, the second tube 2 has a larger diameter than the first tube 1, thereby allowing the first tube 1 to retract into the second tube 2. Extension tubes or telescoping tubes of the prior art exhibited certain limitations in the ability to telescope. Various choices of fittings and tubing materials are available for construction, but combining selected materials and elements with selected dimensions provides the delivery device with a desired rigidity, chemical resistance, and maneuverability for the operator.

The junction point of the first tube 1 and second tube 2 may have a locking mechanism that can be made of a housing 3a and a fitting 3b that can be tightened or loosened by rotating it. The fitting can comprise an o-ring for better sealing and grip, so that the second tube 2 is locked into place when the fitting is tightened. In other embodiments, the locking mechanism comprises a module that auto-detects the proper length and self-locks or tightens when the target length is reached. For example, the monitor can send a signal that causes the fitting to tighten when it reaches a predetermined threshold, such as the length or time and rate the tube is retracted or extended. Prior art devices did not have a suitable fitting and instead either had a rigid non-telescoping contact point, or used connectors with friction to allow a fixed amount of maneuverability. By using a junction that has the proper sealing capacity and adjustability, it has the advantage of accommodating slight variation in tubing dimensions or material, as well as user preference as to the amount of friction or stability wanted with respect to the first and second tubing.

The deployment device can further comprise a third tube 4. In one embodiment, the third tube 4 is coupled to the distal end of the second tube 2. In one embodiment, the second tube 2 is retractable into the third tube 4. In one embodiment the third tube 4 is retractable into the second tube 2. The junction between the second and third tube 4 may comprise fitting that can tighten and/or seal the second and third tube 4 to obtain the preferred stability or locking.

In certain embodiments, the deployment device also comprises, at its distal end, a connector 5, such as a luer fitting, which is optionally associated with a rotatable locking collar. The connector is configured to connect the distal end of the deployment device to an external device, such as a chamber or a cuvette (not shown) in which the sensor-containing distal portion can be stored and/or calibrated. The deployment device can be locked to the chamber by tightening the collar and released from the chamber by loosening the collar.

In one embodiment, the connector 5 comprises a directing element (not shown), such as a valve or leaflet portion, that directs the analyte sensor comprising the optic fiber into different directions. For example, the connector comprises two distal connectors in the shape of a Y-connector. The analyte sensor can be directed by the valve mechanism to proceed and extend from a first distal connector of the Y-connector, for example connected to a storage or calibration chamber. The analyte sensor can be directed by the valve mechanism to proceed and extend from a second distal connector of the Y-connector, for example connected to a cannula.

In one embodiment, the calibration chamber is a tube, for example a glass or plastic tube, that comprises a luer fitting that is configured to engage to the connector 5. The calibration chamber may also comprise a sealing mechanism. The calibration chamber can comprise a heating element, for example a heating coil that can be electrically plugged into the monitor system, wherein the monitor detects a calibration cycle or trigger signal and heats the calibration chamber. The calibration chamber can be a reusable device or a disposable device. The heating element can also be a heating tape wrapped around a plastic tube, which has the advantage of being disposable and manufactured without being excessively expensive. In one embodiment, the calibration buffer is injected directly into the telescoping tube that is housing the chemical indicator system, wherein the seals and connectors in the telescoping tubes allow the distal tube of the telescoping tubes to act as the calibration buffer, wherein heating tape is used to heat the calibration buffer inside the telescoping tube-buffer chamber. In another embodiment, the heating element can be a heating module or oven, wherein the calibration chamber is put into contact, for example by being housed inside the heating module or oven. In yet another embodiment, the heating element will heat the calibration fluid inside the chamber. This has the advantage of allowing the sensor to calibrate at an ideal temperature, for example at physiological temperature in the range of 30˜40° C., such as about 37° C. The heating element can also be configured to heat the calibration buffer to more than one temperature, thereby allowing the analyte sensor to calibration at multiple temperatures.

In one embodiment, the calibration chamber is heated by a heating chamber or holder located on the monitor or separate calibration device. For example, the analyte sensor can be inserted into the calibration chamber comprising the calibration buffer, wherein the entire calibration chamber is placed in contact with a heating element, such as a holder configured to heat, an oven, a water bath, or a heat patch that is wrapped around the calibration chamber. The heating element can be connected to the monitor or calibration controlling module to allow temperature control. The calibration chamber may further comprise a pouch that is connected to the opposite end of the calibration cuvette or tube, wherein the pouch is configured to accept excess or waste calibration fluid or air that is displaced when calibration buffer is injected or ejected from the calibration chamber.

The connector 5, e.g., a luer fitting can also be configured to connect to an indwelling cannula that has already been deployed into the subject (e.g., intravascularly) or to a cannula that will be inserted into the subject, wherein the analyte sensor protected within the telescoping tubes (e.g., after calibration) can be advanced distally into or all the way through the cannula by e.g., sliding the second telescoping tube 2 into the first telescoping tube 3. The deployment device optionally comprises one or more slidable wings that enables the device to be securely attached to the body of the patient after the sensor has been deployed.

Located along the second 2 and/or third 4 tube and concentrically fixed thereto may be a Y junction 6 having an angled port or outwardly extending arm which terminates in a connector 7, said connector optionally configured as a luer fitting, septum, or valve. The side port can also be configured in a Y shape or a T shape. In one embodiment, the junction comprises multiple connectors, for example a three way or four way connecting valve assembly. In another embodiment, the junction further comprises a check valve or a sterile filter.

In one embodiment, the connector 7 is adapted to be connected to a source from which sterile liquid may be introduced into the third tube 4 to surround the analyte sensor comprising the fiber optic line when it is within the tube. Sterile liquid can be introduced to flush the system and remove air bubbles. Also, the angled port may be used for taking blood samples, monitoring blood pressure, providing calibration solution to calibrate the analyte sensor when disposed within the third tube 4, or providing storage solution to store the analyte sensor when disposed within the third tube 4. The connector 7 can also be used to introduce a second sensor, such as the same or different analyte sensor of similar construction, or a reference wire. Thus the extension tube and the associated Y junction allows the user to access the proximal portion of the analyte sensor away from the site of insertion, and deliver or retrieve fluid from the third tube 4 and/or the cuvette, cannula, or patient through the distal luer connector 5. In certain other embodiment, the deployment device comprises a second or third Y junction located at various positions along the first, second or third tube 4, to allow access to various parts.

A cannula protects the site of entry of the analyte sensor comprising the fiber optic line into a blood vessel, for example the radial or femoral artery, or veins. Such protection includes protecting against kinking, bending, etc. during deployment, as well as protecting against various potential breaches in sterility (touching the optical fiber, exposure to the outside air, etc. An optional clamp nut can threadably tighten the Y junction about the second and/or third tube 4 through an o-ring to seal the junction between the second and third tube 4 while allowing the fiber optic line to pass, while the o-ring also optionally acts as a lock for locking the second tube 2 relative to the third tube 4. The fiber optic line in these embodiments may also be or comprise other communication means, such as electrical wires. In another embodiment, the Y junction is locked to the second tube 2 relative to the first tube 1. In one embodiment, the clamp nut has to be loosened to enable the second tube 2 to be moved telescopically with respect to the first tube 1. In certain embodiments, the Y connector comprises a plunger 8 and/or an sealing element 9 or another compressible material.

In these embodiments, the sealing element comprises a hole for the sensor, such as the fiber optic line, to pass through. The hole in the sealing element becomes sealed when it is pressured against a sealing plate of the Y junction, which also comprises a hole for the sensor to pass through, when the plunger is pressed in from tightening the clamp nut. The use of the compressed fitting has the advantage over the prior art sealing mechanism in that it tightly seals and is durable, without the risk of chemical leaching that results when chemical adhesives are used. The sealing element may further comprise coating to enhance the sealing and chemical resistance. The plunger and sealing element also have less leakage problems from movement of the optical fiber during telescopic movement of the tubing, because the amount of wear and thermal damage to the fiber and seal can be minimized by decompressing the fitting.

The tubing may further comprise gradations 11, preferably in cm, to enable the operator to determine the depth of penetration when the sensor is inserted in a patient's blood vessel. When the second tube 2 is in a fully extended or partially extended position relative to the first tube 1, a portion of the second tube 2 between the clamp nut and the distal end of the first tube 1 is exposed and this exposed portion preferably has gradations. The gradation may alternatively be on the first tube 1 or third tube 4, where the retracting tube is the first or third tube 4 relative to the second tube 2. In one embodiment, the third tube 4 comprises a chemical coating. The chemical coating may comprise various polymers known in the art, such as heparin coating, anti-biotic coatings, anti-inflammatory coatings, insulin coatings, microbial coatings, or antioxidant coatings.

In one embodiment, the exposed portion of the second tube 2 comprises a removable stop which facilitates positioning of the sensor when it is inserted in a patient's radial artery. In certain embodiments, the sensor comprises optical fibers and electrical wires, which are connected to a connector terminal comprising sockets and ferrules. The connector can further comprise connection for the temperature sensor on the sensor assembly, as well as an EEPROM to store information, and a clip to bind the tubing to store the entire assembly in a compact fashion. A temperature sensor can also be located on the Y-junction or within the third tube 4, to measure the temperature of any fluid within the third tube 4 or being flushed.

The delivery device can further comprise a pressure sensor to detect any negative or positive pressure within the device. In one embodiment, the delivery device has a pressure diffusing mechanism when it detects a positive or negative pressure buildup that exceeds a predetermined threshold. The device can also comprise a temperature sensor or use the temperature sensor on the analyte sensor to measure the temperature of the fluid inside the third tube 4 and in contact with the sensor.

In one embodiment, the delivery device comprises a roller module that rotatably wraps the optical fiber around a winder. In this embodiment, the optical fiber can be extended or retracted without having to telescope a tube, but rather rotating a winder that has the optical fiber wound around it. In this embodiment, the delivery device comprises the roller module attached to a proximal end of a single tube, wherein the junction of the roller module and single tube comprises a Y-junction, similar to a one discussed above. The distal end of the single tube comprises a connector to be engaged with an external device, for example a calibration chamber or cannula.

### Latch/Base Mechanism

FIG. 2A and FIG. 2B illustrate an embodiment of a latch mechanism 60 having a latch or clip portion 11, a housing 59, and base portion 12 that can be used as a locking mechanism in certain embodiments of the deployment device. The latch portion 11 and the housing 59 can be made into a subassembly. FIG. 3A shows one embodiment wherein the base 12 is located at the proximal end of the third tube 61 and the subassembly made from the latch 11 and housing 59 is located at the distal end of the first tube 62. The base 12 also has a side port 63 that connected to a tubing 64. In one embodiment, the latch 11 and base 12 will engage with each other into an locked state shown in FIG. 2A and FIG. 3A, for example by a snap-in mechanism.

FIG. 2B shows the mechanism in the open state. The locking can prevent the tubes from rotating, thereby improving usability. In one embodiment, latch mechanism 60 when in the closed or engaged form has a flat shape, thus making it easier to secure to the subject and also being better suited for attaching latch mechanism 60, for example by tape or adhesive, to the subject. The deployment device can also include a device holder, which is capable of securing a portion of the deployment device such as latch mechanism 60. In one embodiment, the device holder has an adhesive backing suitable for use in a medical environment and thus inhibits unintentional removal or movement of the optical sensor. By having an adhesive backing, there is less risk of circumferential compression to the patients arm. In one embodiment, the latch mechanism 60 has an adhesive backing, such that no additional device holder is required to secure the deployment system onto the patient.

In one embodiment, the latch mechanism 60 has curved or smooth surface such that it does not have any sharp or rigid corners. Such a design has the advantage of reducing the pressure point to the patient when the latch mechanism 60 is optionally attached to the subject, such as a patient's arm. In one embodiment, the latch mechanism 60 has a layer with a softer texture, such as foam, silicone, rubber, vinyl, or other polymer coating or casing, so that it reduces the pressure to the patient.

FIG. 3B illustrates a cross-section view of the latch/base portion. In one embodiment, the junction point of the first tube 62 and second tube (not shown) has a latch 11 and base 12 portion. In one embodiment the latch assembly comprises a slide portion 13 that can receive the first tube that is sealed by a barb or thread fitting, and the housing 59 houses the slide portion 13. The latch comprises a hollow channel 65 in the middle for the fiber optic to pass through. In one embodiment, the latch comprises a washer 14 and/or a gasket 15 to allow the latch mechanism 60 to maintain a seal so that fluid will not enter the first tube 62 or second tube, for example when calibration solution is optionally entered into the second tube. This both maintains a better sterile seal and allows buffer to be kept inside the tube area, for example for calibration or so that buffer does not leak down the tube to areas that should not be in contact with liquid, for example areas that are exposed to electrical wires.

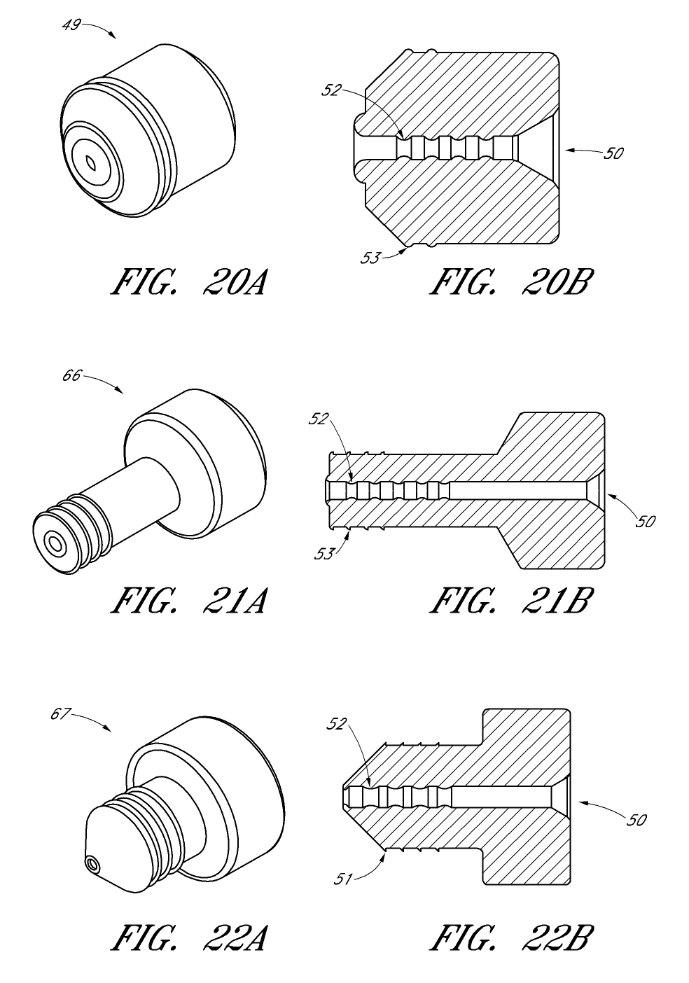

The deployment device can have a static seal and/or a dynamic seal that seals the area from fluid from passing through. The washer 14 and/or gasket 15 can be used to accomplish this. In one embodiment, the base portion 12 has a seal from a static crush gasket, wherein the seal is open until the user clips the latch 11 with the base 12 together. The washer 14 and gasket 15 provides an additional press fit seal when the press 51 is pushed in by a portion of the latch 11. In one embodiment, the latch portion has a seal from a dynamic seal that maintains a seal when the optical fiber is moving through the hollow tube, and then can be crushed or pressed like the static seal. The washer 14 and gasket 15 provides a seal even when the latch 11 and base portion 12 are not engaged, and the additional press fit provides increased or improved sealing between the fiber optic and the hollow tube portions. In one embodiment, the dynamic seal is made by putting an O-ring in front of a static seal. FIG. 20A illustrates one embodiment of a gasket 49 that can maintain a dynamic seal and a static seal. FIG. 20B illustrates that the internal opening 50 of the gasket has several rib elements 52 in the internal surface of the gasket that maintain a seal between the rib elements 52 and the optical fiber that is placed through the gasket. Upon application of pressure, for example by having the press 51 apply pressure when the latch 11 engages the base 12 as shown in FIG. 3B, a static seal is added to ensure the seal between the gasket's inner surface and the optical fiber passing through. The gasket may also have ribs 53 on the outer surface so that the gasket is securely placed within the base 12 or other housing structure of the deployment device. FIG. 21A and FIG. 21B illustrate another embodiment of the gasket 66 with a longer length and a section with a first diameter and a section with a second diameter. This embodiment also can have a dynamic and static seal function. This embodiment also has an opening 50 with several rib elements 52 in the inner surface and ribs 53 on the outer surface. FIG. 22A and FIG. 22B illustrate another embodiment of the gasket 67 with a longer length and a section with a first diameter and a section with a second diameter. This embodiment also can have a dynamic and static seal function. This embodiment also has an opening 50 with several rib elements 52 in the inner surface and ribs 53 on the outer surface.

In certain embodiments, the latch 11 and housing 59 assembly portion can be located at the distal end of the first tube 62, or at the junction of the first tube 62 and second tube. The base 12 is located at the proximal end of the third tube 61. In one embodiment, the latch/base portion or a tube connector location is adjustable along the telescoping tube, for example by a fastening member or a locking system. In a preferred embodiment, the base 12 is at a predetermined position, thereby allowing the user to place the sensor portion into a predetermined deployment position when the latch 11 is engaged with the base 12. This also can have the advantage of reducing accidental damage to the subject or the device, because the optical fiber cannot extend beyond the predetermined position accidentally. In this embodiment, because the deployment position will allow the sensor portion to extend from the distal portion of the third tube by a fixed length, which is the length ideal for deployment into for example a predetermined artery or vein, the operator does not need to conduct the step of monitoring and adjusting the amount of the sensor portion to extend. This has the advantage of improved usability. The length of the various tubes, the length of the fiber optic, and the subject location to be measured are factors to consider.

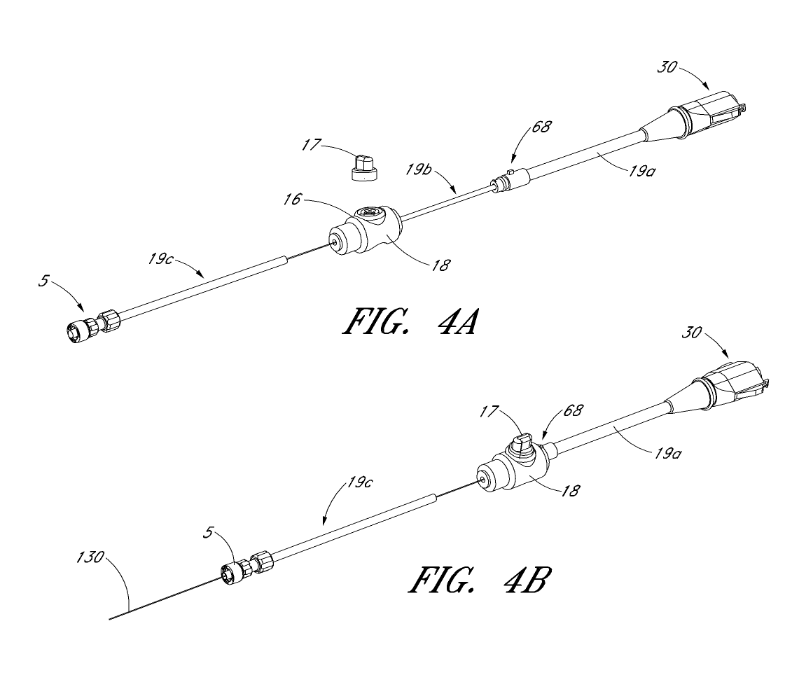

FIG. 4A and FIG. 4B illustrates another embodiment wherein a base portion 18 has a lock 16 and key 17 mechanism. The lock and key mechanism is used to secure or hold the first tube 19a when the locking housing 68 located on the distal end of the first tube 19a engages with the base portion 18. The key 17 is optionally removable, such that the user or patient will not accidentally unlock the mechanism. On the proximal end of the deployment device is the cover 30 for the optical/electrical sensor connector. FIG. 4A shows the telescoping tubes in an extended configuration wherein the first tube 19 is distant from the base portion 18 and the second tube 19b is exposed. FIG. 4B shows the telescoping tubes in a contracted configuration wherein the first tube 19 is engaged with the base portion 18. In this configuration, the optical fiber 130 is extended from the connector 5 of the third tube 19c such that it enters the subject. In one embodiment, the length of the optic fiber, the location of the base, and the length of the various tubes creating the telescoping tubes have respective lengths so that the amount the optical fiber extends from the connector 5 such that by a fixed length, which is the length ideal for deployment into for example a predetermined artery or vein.

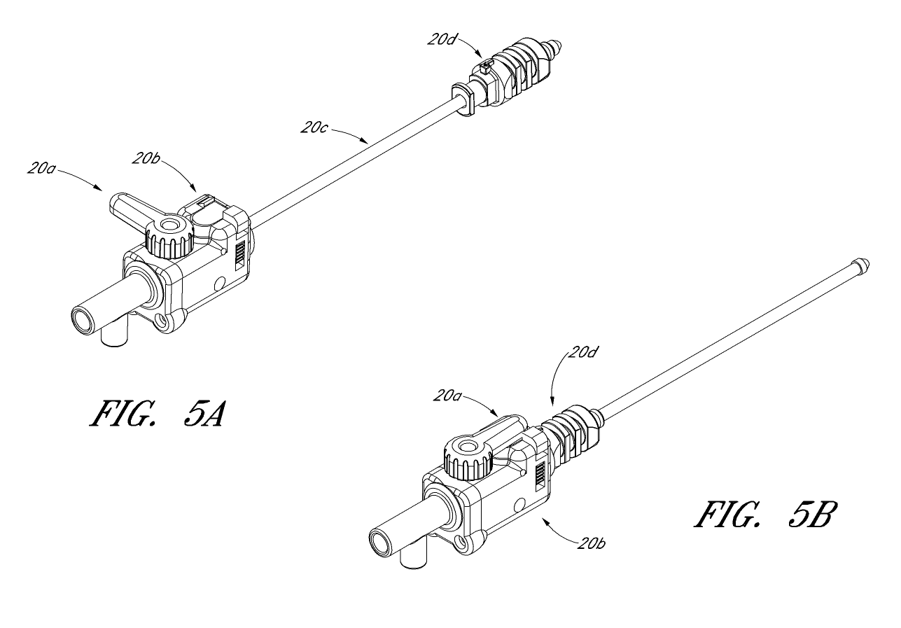

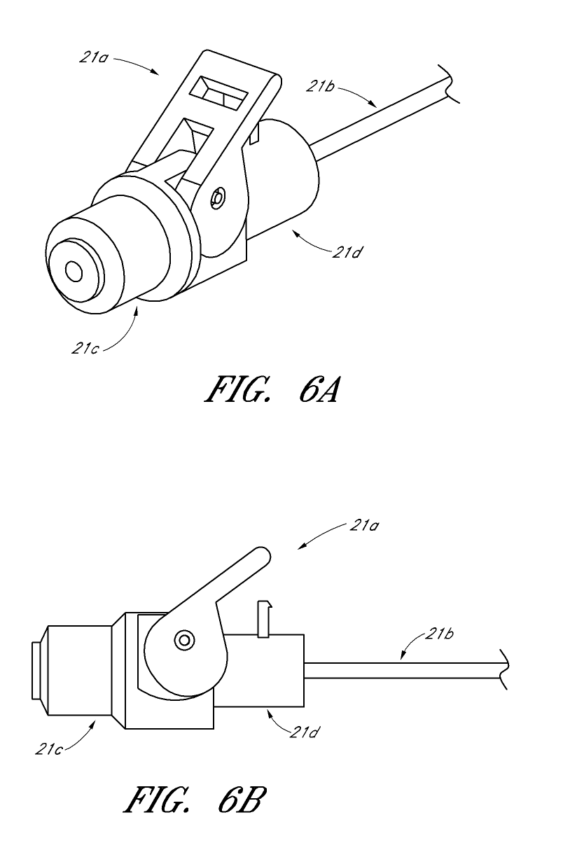

FIG. 5A and FIG. 5B illustrates another embodiment wherein a base portion 20b has a lock 20a. FIG. 5A shows the telescoping tubes in an extended configuration where the locking housing 20d is separate from the base portion 20b and shows the second tube 20c. FIG. 5B shows the telescoping tubes in a contracted configuration where the locking housing 20d is engaged with the base portion 20b and the lock 20a is locked. FIG. 6A and FIG. 6B illustrate another embodiment wherein a clipping mechanism 21a is used to fasten the telescoping tubes in the contracted configuration. In this embodiment, the base portion 21c is located at the proximal end of the third tube (not shown) and the locking housing 21d is located at the distal end of the first tube 21b. When the locking housing 21b is engaged with the base portion 21c as shown, the clipping mechanism 21a is clipped in to secure the locking housing 21d to the base portion 21c. In these various embodiments, the base portion is at a predetermined position such that when the latch or similar portion is engaged with the base portion, the optical fiber having the chemical indicator system is extended by a predetermined length so that it extends by the appropriate amount depending on the subject or the location to be measured. In another embodiment, the base portion comprises a fastener that allows the position of the base portion to change along the telescoping tubes. This embodiment allows the user to make adjustments to the amount the optical fiber extends from the distal end of the deployment device, for example when the predetermined extension amount is too long or too short for a desired application. In yet another embodiment, the latching and locking mechanism can have both mechanisms that allow a predetermined deployment amount and the manual adjustment to make any additional changes in addition to or in instead of the predetermined deployment amount.

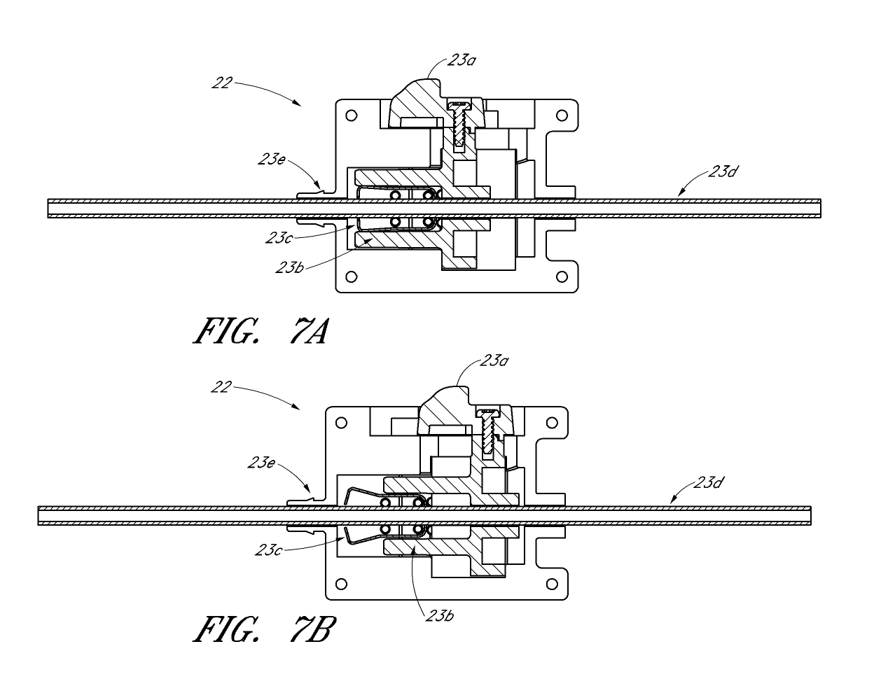

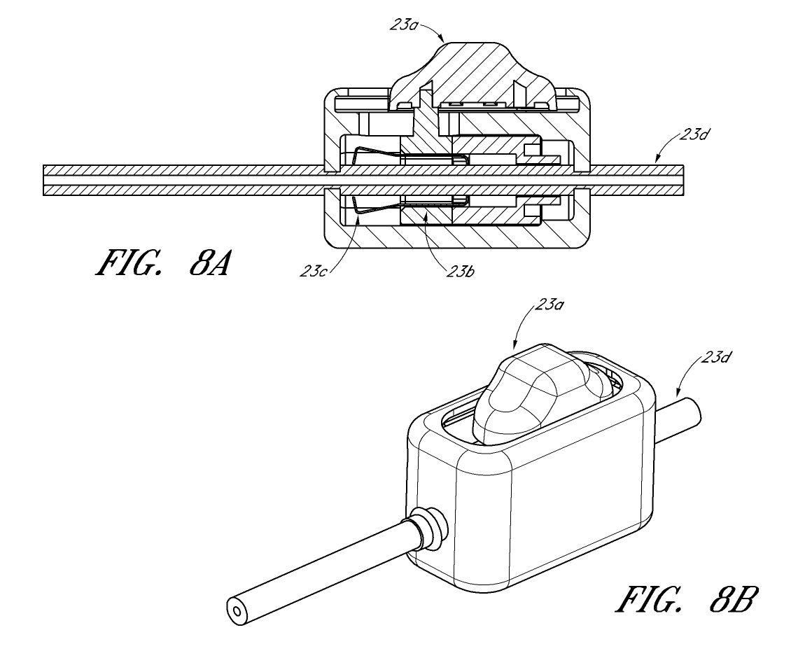

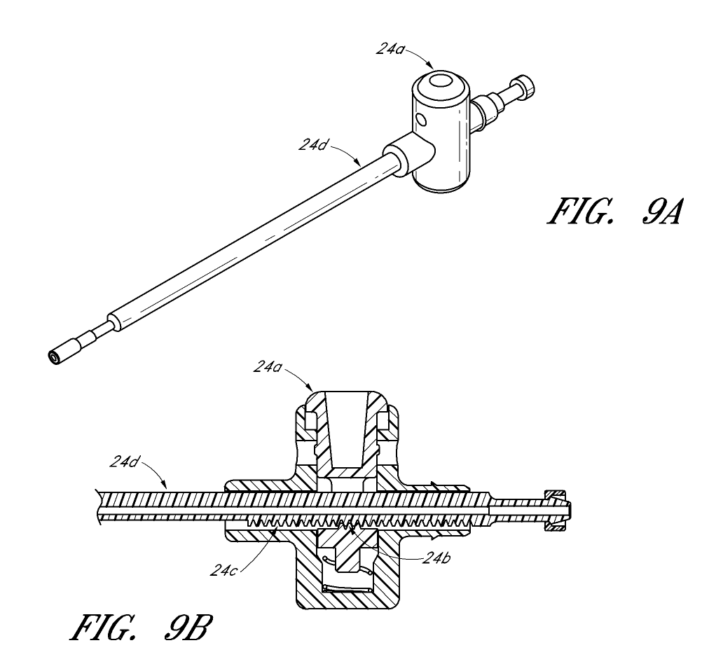

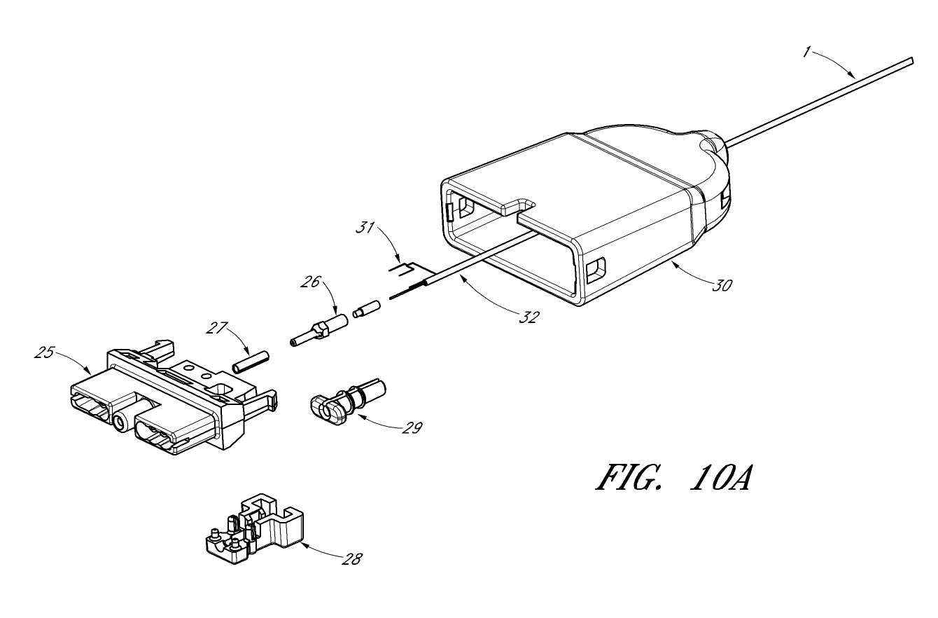

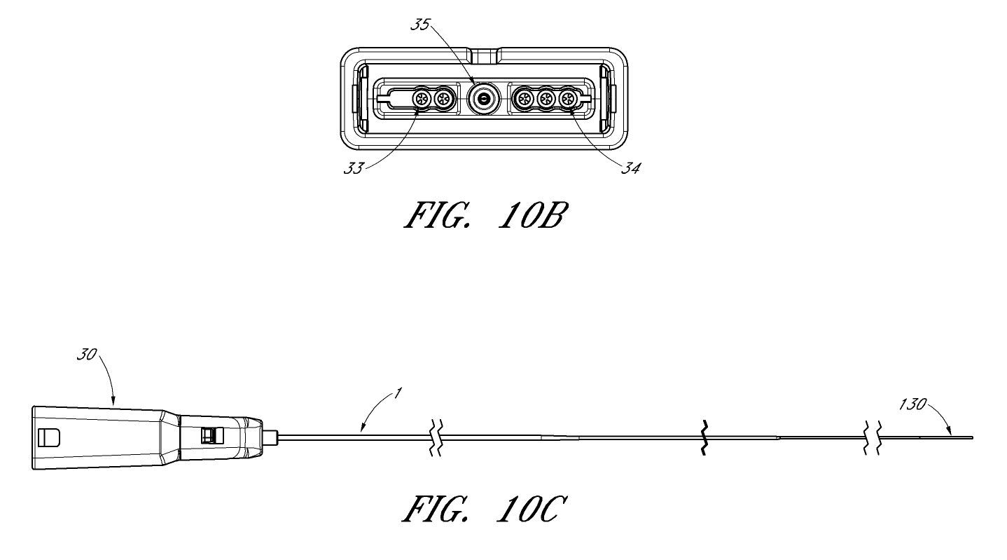

FIG. 7A and FIG. 7B illustrates an embodiment wherein the telescoping tubes are controlled by an operably connected adjustment module 22 that can adjust the amount the distal tube is retracted or extended. The adjustment module 22 is attached to the second tube 23d and the proximal end of the adjustment module 22 connects to the first tube (not shown) using a barb fitting 23e. The adjustment module has a button 23a that is slidable such that in the open position it can be moved and thereby adjusting the amount of telescoping. FIG. 7A shows the adjustment module 22 in the closed position, where the button 23a is slid so that the plate 23c moves onto the spring grip element 23c. The spring grip element 23c then grips onto the second tube 23d such that it does not move easily. Thus when the button is in the close position, the adjustment module is fixed and thus the amount of telescoping is fixed. FIG. 8A and FIG. 8B illustrate another embodiment wherein an operably connected adjustment module can freely adjust the amount of retraction or extension. This embodiment also has a similar button 23a, a plate 23c, and a spring grip element 23c that can grip onto the second tube 23d when in the closed configuration. FIG. 9A and FIG. 9B illustrates yet another embodiment wherein an operably connected adjustment module can adjust the amount of retraction or extension, wherein the adjustment module comprises a button 24a that allows the adjustment module to be in the open or closed position. In this embodiment, when the operator presses the button 24a down, it will release the teeth 24b from the grooves 24c in the rack 24d.

In one embodiment, the telescoping tubes of the deployment device are in the fully extended configuration during storage and shipment, thus the optical fiber is completely housed inside the hollow tubes of the deployment device. In one embodiment, e.g., during calibration within the deployment device, the telescoping tubes of the deployment device may be kept in their extended state, thus allowing a calibration buffer to be directly introduced into the telescoping tube, for example the distal third tube, wherein the chemical indicator system along the distal end region of the optical fiber sensor will still be completely retracted within and housed inside the telescoping tubes, such that the chemical indicator system will be in contact with the calibration buffer inside a calibration chamber that is formed within the distal third tube. In another embodiment, e.g., where calibration occurs in an external calibration chamber, the telescoping tubes of the deployment device may be changed to the retracted state, thus the chemical indicator system on the optical fiber is extended outside the telescoping tubes, thereby allowing the chemical indicator system to enter a calibration chamber, that is preferably coupled to the distal connector of the deployment device.

### Cable Connectors

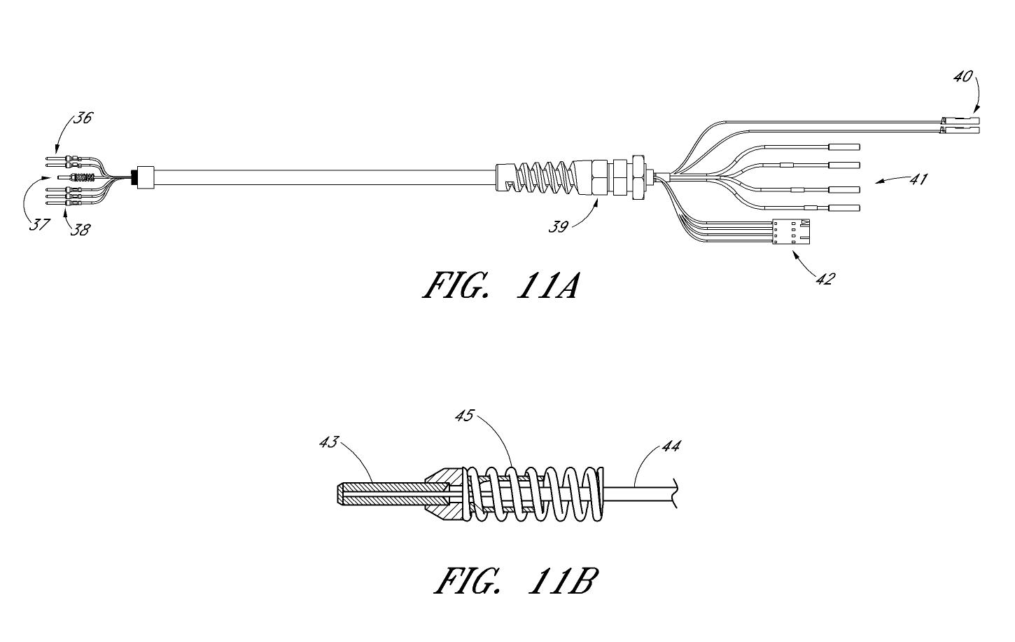

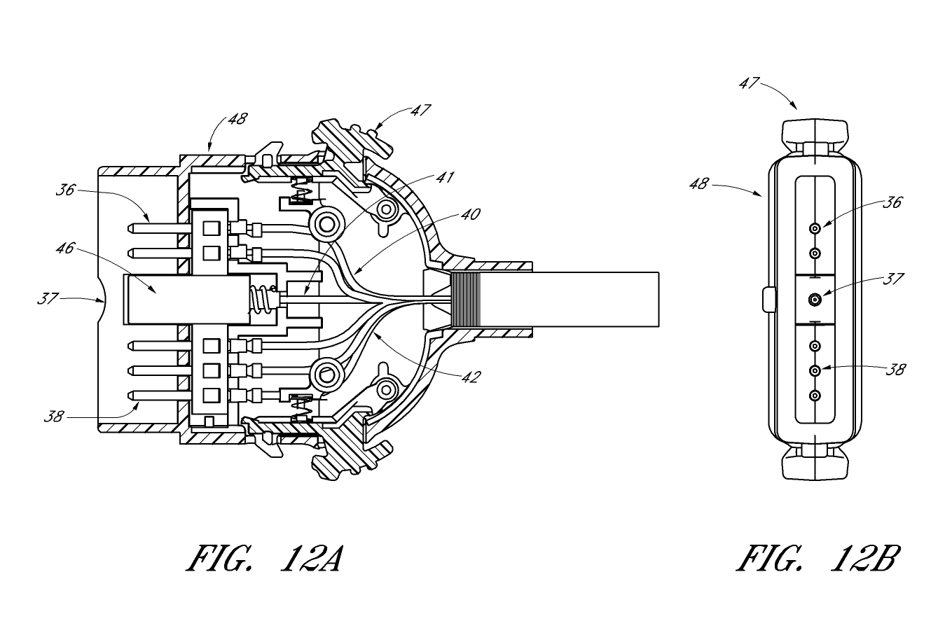

FIG. 10A is an optical/electrical sensor connector located at the proximal end of the analyte sensor. In one embodiment the connector is configured to both allow electrical communication and optical communication between the monitoring system that has a display function and the light emitting source, or optionally the module that comprises similar functions. In one embodiment the connector has a coupling element 25 that houses the connecting elements, such as the optical ferrule 26, thermocouple, or electrical wires. In one embodiment, the optical ferrule 26 is engaged with a split sleeve 27 and a ferrule capture element 28. The connector can further comprise a tube adapter 29. In one embodiment the connector comprises a memory module and/or a memory accessing slot, for example a Serially Electrically-Erasable Programmable Read-Only Memory (SEEPROM), other nonvolatile memory modules, including RFID memory systems. The various components are housed inside a cover 30 that allows the proximal first tube to be joined, including a thermocouple 31 and optical fiber tubing 32. The cover 30 can also have an opening to allow a latch on the mating monitor cable connector to engage.

FIG. 10B is a front view of the connector's proximal end that couples with the connector or module from the monitor system side. In one embodiment, the optical ferrule 35 is housed in the middle section to preserve maximum stability and minimum noise when external force is applied to the connector assembly, or when the sensor and/or cables are accidentally or intentionally moved during use. In other embodiments, there can be multiple optical ferrules. The thermocouple 33 can be placed on one side of the optical ferrule connector 35, while the electrical wire connector 34 are on the other side of the optical ferrule connector 35. In other embodiments there can be a single thermocouple and a single electrical wire connector, whereas in others there can be multiple thermocouples and wire connectors. FIG. 10C is a side view of the deployment device with the connector. The connector has a cover 30 and tubing 1, with the most distal end having the optical fiber exposed depending on the telescoping tube configuration.

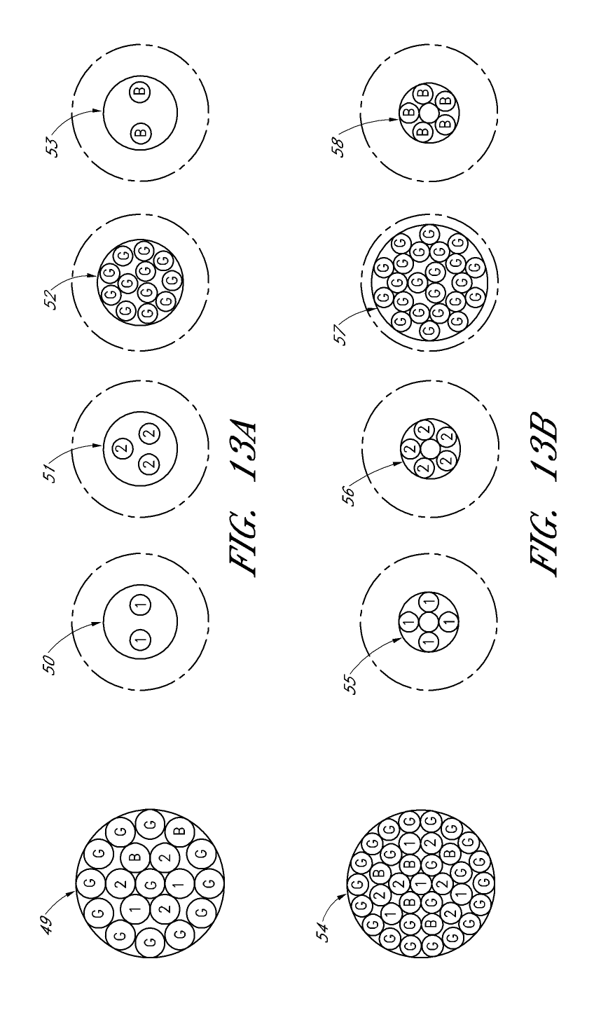

The present analyte sensing system can further have a monitor cable as shown in FIG. 11A that houses the optical and electrical wires that connect the analyte sensor having the chemical indicator system with the monitor or module that has the LEDs and/or emission detectors and a processor to execute various operations. In one embodiment, the monitor cable has a thermocouple 36, an optical ferrule 37, and electrical connectors 38 that are configured to connect to the sensor connector coupling element 25. The various wires and connectors can be a single wire or connector, or a plurality of wires or connectors. In one embodiment the connector has a strain relief 39 to add durability to the cable, which can optionally be on the proximal and distal end of the monitor cable. The monitor cable also comprises thermocouple wires 40, optical bundles 41, and electrical wires 42, which are configured to connect to the system display monitor or a intermediate module that comprises the LEDs and detectors, and a processor to execute various operations. FIG. 11B is a magnified view of the optical ferrule 37 connected to the fiber bundles 44. In one embodiment, the optical ferrule comprises a crimp housing 43 for stable and secure input into an optical ferrule holder. The optical ferrule can further comprise a spring element 45 to add stability and other bonding materials for assembly.

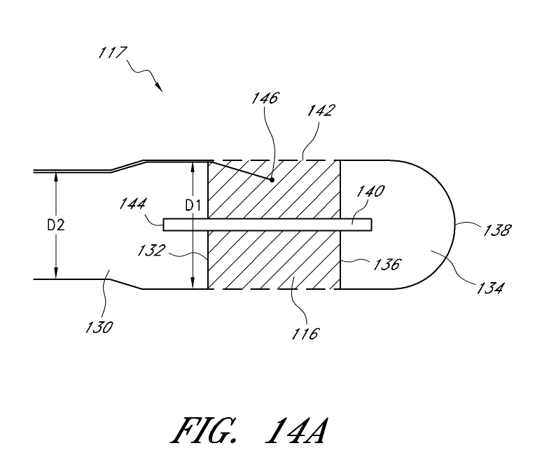

FIG. 12A is an inside view of a monitor cable with the various components assembled onto the monitor cable connector shell or housing 48. In one embodiment, the connector shell 48 has a pin holder 46 that will hold the various connectors and wires. The pin holder has a portion for the thermocouple 36, optical ferrule 37, and electrical wires 38. The monitor cable connector shell can also have a latch button 47 that allows the monitor cable connector to attach the sensor connector shown in FIG. 10A through 10B. The thermocouple wires 40, optical bundles 41, and electrical wires 42 are shown to come through from the cable. FIG. 12B is a view from the distal end of the monitor cable, showing the various components of the monitor cable connector. These various connectors, both from the sensor side and the monitor cable side, have the advantage of being robust to movement, especially with regards to the optical communication through the optical fibers and optical ferrule. Both the shape of the connectors, the arrangement of the connecting elements, as well as the pin holder 46 and the optical ferrule 37 contribute to the robustness and associated reduction of noise as a result of mechanical disruption to the sensors and cable and associate connection.

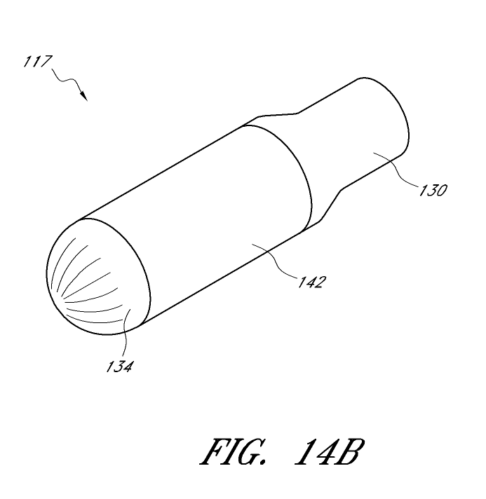

FIGS. 13A and 13B illustrates certain embodiments wherein the optical fibers are systematically mapped among several sensors and several detectors. The optical fibers can be divided and mapped into a single cable in various ratios, for example, 4:1, 9:1, 19:1, or 40:1. In a preferred embodiment, the ratio is 37:1. In a more preferred embodiment, the ratio is 19:1. In one embodiment, the optical cable comprises between 15 and 50 optic fibers, each fiber being between 20-60 μm in diameter. In a preferred embodiment, the optic cable comprises 37 optical fibers, each fiber being 35 μm in diameter. In a more preferred embodiment, the optic cable comprises 19 optical fibers, each fiber being 50 μm in diameter. Optical fibers of different sizes and numbers can be utilized. In one embodiment, the fibers are coupled to a single sensor. In systems comprising multiple sensors, the fibers may be split systematically between the several sensors, for example 3 or 4 sensors. In one embodiment, the coupling of the fibers, for example a 19:1 or 37:1 ratio fiber is coupled to a single glass or plastic optical fiber. The coupling can comprise various materials and methods known to one skilled in the art.

In one embodiment, the orientation of the fibers is such that specific fibers are mapped for the efferent blue signals and likewise a number of fibers mapped for the afferent green and blue signals. Mapping the different optical fibers can achieve signal separation. In one embodiment, this is accomplished by having the “launch” light fibers relative to detection fibers. Splitting the optical fibers between emitters and detectors and various wavelengths that are needed can improve signal-to-noise ratio by reducing the instances of optical interference, leakage, loss, or attenuation.

By systematically mapping the different fibers, a uniform excitation and emission is achieved to optimize the signals, for example by reducing signal variation and noise associated with illuminating and/or detecting non-uniform light. In other embodiments, the optical fibers are randomized in order to emit a substantially uniform emission. In other embodiments, an optical diffuser is used to emit a substantially uniform emission. Such optical adjustment elements can be also used in the emission from the sensor chemistry.

In one embodiment, the fibers are broken out at the proximal ends into four different ferrules, each ferrule which mates to either one of the two LEDs or one of the two detectors. In a preferred embodiment as shown in FIG. 13B, a first LED uses 4 fibers 55, a second LED uses 5 fibers 56, whereas the signal is detected using 23 fibers 57 coupled to a first detector and the reflected reference signal are coupled to a second detector using 5 fibers 58. This yields a total of 37 mapped fibers 54 illuminating the chemical indicator system. In a more preferred embodiment, a first LED uses 2 fibers 50, a second LED uses 3 fibers 51, whereas the signal is detected using 12 fibers 52 coupled to a first detector and the reflected reference signal are coupled to a second detector using 2 fibers 53. This yields a total of 19 mapped fibers 49 illuminating the chemical indicator system. For systems employing more than two detectors and/or emitters, the number of fibers and their proportional arrangements can be adjusted. This particular fiber mapping is an example of one mapping format; other mappings may be used to match new sensor designs which require more or less light or different wavelengths of light for enhanced functionality.

### Sterile Delivery System

The delivery device also allows the handling and deployment of the analyte sensor without compromising the sterility or sensor integrity, such as damages from dehydration or irradiation from light. Certain analyte sensors may be unstable when exposed to the environment, for example due to poisoning from oxidation or other atmospheric chemicals.

In one embodiment, the sensor is prepared and handled so as to maintain sterility and protect the sensor from dehydration or other environmental factors. The analyte sensor is manufactured and packaged sterile. In certain embodiments, the analyte sensor comprises a storage cuvette comprising a storage solution. The storage cuvette can comprise a sealing mechanism similar to the Y junction described above, by having a connector with a clamp rod plunger with a hole and a sealing element with a hole. The hole can allow the communication wire, such as the fiber optic line, to pass through.

The user opens the sensor and removes the storage cuvette comprising a storage solution from the distal luer, while keeping the second tube to the fully extended position so the sensor is not exposed and is inside the third tube. The calibration chamber with the calibration solution is attached to the distal luer. After attaching the distal end of the deployment device to the calibration chamber, the analyte sensor is pushed into the chamber by moving the second tube to the retracted position or vice versa, which ever results in a configuration wherein the analyte sensor is extending. The user may optionally flush the third tube that houses the sensor with sterile fluid or calibration fluid through the connector on the Y junction. Alternatively, the calibration may be conducted by adding calibration fluid into the third tube with the analyte sensor therein. This allows the user to calibrate the analyte sensor while avoiding exposure to the environment.

In another embodiment, the calibration chamber constitutes the storage cuvette and is shipped pre-attached. In such embodiments, the calibration solution and storage solution can be the same solution. In the embodiment where the storage solution is different, the calibration solution is injected into the pre-attached chamber and the storage solution is displaced into the waste pouch that is attached to the calibration chamber at the distal end or the proximal end through a three way valve. The pouch may further comprise a device that allows the pouch to actively aspirate the solution out of the chamber or cuvette.

After calibration the locking mechanism and/or the clamp nut are loosened so that the calibration chamber may be removed and the second tube be extended forward to envelop the distal portion of the analyte sensor. Sterile liquid introduced through the connector 7 flushes out the calibration solution, removes air bubbles, maintains a sterile environment around the analyte sensor and prevents contamination from atmospheric contaminants. In certain embodiment, heparinized saline solution is introduced to prevent clot formation. In other embodiments, the device can be configured to deliver therapeutic agents, such as antibiotics, insulin, antioxidants, or other preservatives. The apparatus may be locked in this position with the catheter retracted inside the deployment device by tightening the fitting 3. When the catheter is to be introduced into a patient, the distal connector is connected to a cannula, previously inserted into the subject, the optional clamp nut is loosened and the second tube is retracted back into the first tube thereby allowing the sensor comprising the optical fiber to be threaded into the cannula.

The analyte sensor may be inserted in various lumens of the patient. In certain embodiments, the first tube, second tube, and third tube, as well as the fiber optic cable for the analyte sensor have lengths to achieve the required length needed to reach the target patient lumen. In one embodiment, the delivery device is designated for an arterial delivery, and comprises a third tube that is about 1.5 inches, while the first tube is about 9.5 inches. In one embodiment the delivery device is designated for a venous delivery, and comprises a third tube that is about 3.0 inches, while the first tube is about 4.2 inches. In other embodiments, the venous lumen accessing configuration comprises the fiber optic cable and the second tube to be about twice the length compared to an artery accessing configuration. The length may also be adjusted according to the patient type, such as pediatric lengths. The device deployment length may also be adjusted based on the type and length of the catheter to be used in conjunction.

In one embodiment, the optional removable stop is placed at a predetermined distance along the second tube and the catheter is inserted to a depth so that the stop rests against the distal end of the first tube, so it reaches the proper depth for the target lumen, for example the radial artery position. In one embodiment, the stop is removed and the second tube is retracted so that the end of the clamp nut comes to rest against the distal end of the first tube, so it reaches further, for example the femoral artery position, since patients are of different sizes, the operator may determine the proper depth of insertion by using the gradations on the second tube. When the catheter is inserted to the proper position, the stopper or clamp can be tightened, thus locking the deployment device, while the apparatus is strapped or taped to the patient with the optional aid of the wings.

In summary, the apparatus comprising the combination of catheter and deployment device facilitates the introduction of the catheter into a blood vessel through a cannula while minimizing contamination by physical contact. The deployment device comprises: (i) the extension tube which allows the fixing of various clinical tubing fittings to be distal from the site of cannulation, (ii) the Y junction compression fitting which allows reversible hermetic sealing around the communication wire. The Y junction also allows the attachment of pressure lines, blood sampling lines, solution injection lines and other accessories, (iii) the concentric first, second, and third tubes allow the advancement of the catheter by sliding of the second tube attached to the catheter relative to the first while keeping the analyte sensor completely covered. Thus, when in the advanced position, no portion of the sensor or optical fiber that may make contact the body fluids will have been contaminated or damaged by exposure. In other embodiment, the space surrounding the sensors within the catheter is filled with a hydrophilic medium, and the cells in the optical fiber sensors are filled with an indicator-containing medium.

### Glucose-Sensing Chemical Indicator Systems

In certain embodiments, the hydrogels are associated with a plurality of fluorophore systems. In certain embodiments, the fluorophore systems comprise a quencher with a glucose receptor site. In certain embodiments, when there is no glucose present to bind with the glucose receptor, the quencher prevents the fluorophore system from emitting light when the dye is excited by an excitation light. In certain embodiments, when there is glucose present to bind with the glucose receptor, the quencher allows the fluorophore system to emit light when the dye is excited by an excitation light.

In certain embodiments, the emission produced by the fluorophore system may vary with the pH (as well as the temperature) of the solution (for example, blood), such that different excitation wavelengths (one exciting the acid form of the fluorophore and the other the base form of the fluorophore) produce different emissions signals. In preferred embodiments, the ratio of the emission signal from the acid form of the fluorophore over the emission signal from the base form of the fluorophore is related to the pH level of the blood. In certain embodiments, an interference filter is employed to ensure that the two excitation lights are exciting only one form (the acid form or the base form) of the fluorophore. Chemical indicator systems, hardware configurations and methods for determining both pH and glucose based on ratiometric determination are described in detail in U.S. Pat. No. 7,751,863 and co-pending U.S. application Ser. Nos. 12/027,158 (published as 2008/0188725) and 12/612,602; incorporated herein in their entirety by reference thereto.

The indicator system (also referred to herein as a fluorophore system) can comprise a fluorophore operably coupled to a quencher. In certain embodiments, the fluorophore system comprises a polymer matrix comprising a fluorophore susceptible to quenching by a viologen, a viologen quencher with quenching efficacy dependent on glucose concentration, and a glucose permeable polymer, wherein said matrix is in contact with blood in vivo. Preferably the fluorophore is a fluorescent organic dye, the quencher is a boronic acid functionalized viologen, and the matrix is a hydrogel.

“Fluorophore” refers to a substance that when illuminated by light at a particular wavelength emits light at a longer wavelength; i.e. it fluoresces. Fluorophores include but are not limited to organic dyes, organometallic compounds, metal chelates, fluorescent conjugated polymers, quantum dots or nanoparticles and combinations of the above. Fluorophores may be discrete moieties or substituents attached to a polymer.

Fluorophores that may be used in preferred embodiments are capable of being excited by light of wavelength at or greater than about 400 nm, with a Stokes shift large enough that the excitation and emission wavelengths are separable by at least 10 nm. In some embodiments, the separation between the excitation and emission wavelengths may be equal to or greater than about 30 nm. These fluorophores are preferably susceptible to quenching by electron acceptor molecules, such as viologens, and are resistant to photo-bleaching. They are also preferably stable against photo-oxidation, hydrolysis and biodegradation.

In some embodiments, the fluorophore may be a discrete compound.

In some embodiments, the fluorophore may be a pendant group or a chain unit in a water-soluble or water-dispersible polymer having molecular weight of about 10,000 daltons or greater, forming a dye-polymer unit. In one embodiment, such dye-polymer unit may also be non-covalently associated with a water-insoluble polymer matrix M¹ and is physically immobilized within the polymer matrix M¹, wherein M¹ is permeable to or in contact with an analyte solution. In another embodiment, the dye on the dye-polymer unit may be negatively charged, and the dye-polymer unit may be immobilized as a complex with a cationic water-soluble polymer, wherein said complex is permeable to or in contact with the analyte solution. In one embodiment, the dye may be one of the polymeric derivatives of hydroxypyrene trisulfonic acid. The polymeric dyes may be water-soluble, water-swellable or dispersible in water. In some embodiments, the polymeric dyes may also be cross-linked. In preferred embodiments, the dye has a negative charge.

In other embodiments, the dye molecule may be covalently bonded to the water-insoluble polymer matrix M¹, wherein said M¹ is permeable to or in contact with the analyte solution. The dye molecule bonded to M¹ may form a structure M¹-L¹-Dye. L¹ is a hydrolytically stable covalent linker that covalently connects the sensing moiety to the polymer or matrix. Examples of L¹ include lower alkylene (e.g., C₁-C₈ alkylene), optionally terminated with or interrupted by one or more divalent connecting groups selected from sulfonamide (—SO₂NH—), amide —(C═O)N—, ester —(C═O)—O—, ether —O—, sulfide —S—, sulfone (—SO₂—), phenylene —C₆H₄—, urethane —NH(C═O)—O—, urea —NH(C═O)NH—, thiourea —NH(C═S)—NH—, amide —(C═O)NH—, amine —NR— (where R is defined as alkyl having 1 to 6 carbon atoms) and the like, or a combination thereof. In one embodiment, the dye is bonded to a polymer matrix through the sulfonamide functional groups.

In one preferred embodiment, the fluorophore may be HPTS-CysMA (structure illustrated below); see U.S. Pat. No. 7,417,164, incorporated in its entirety herein by reference thereto.

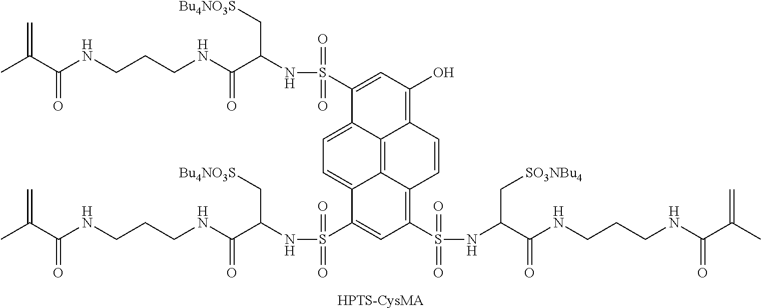

Of course, in some embodiments, substitutions other than Cys-MA on the HPTS core are consistent with aspects of the present invention, as long as the substitutions are negatively charged and have a polymerizable group. Either L or D stereoisomers of cysteine may be used. In some embodiments, only one or two of the sulfonic acids may be substituted. Likewise, in variations to HPTS-CysMA shown above, other counterions besides NBu₄⁺ may be used, including positively charged metals, e.g., Na⁺. In other variations, the sulfonic acid groups may be replaced with e.g., phosphoric, carboxylic, etc. functional groups.

Fluorescent dyes, including HPTS and its derivatives are known and many have been used in analyte detection. See e.g., U.S. Pat. Nos. 6,653,141, 6,627,177, 5,512,246, 5,137,833, 6,800,451, 6,794,195, 6,804,544, 6,002,954, 6,319,540, 6,766,183, 5,503,770, and 5,763,238; each of which is incorporated herein in its entirety by reference thereto.

In accordance with broad aspects of the present invention, the analyte binding moiety provides the at least dual functionality of being able to bind analyte and being able to modulate the apparent concentration of the fluorophore (e.g., detected as a change in emission signal intensity) in a manner related to the amount of analyte binding. In preferred embodiments, the analyte binding moiety is associated with a quencher. “Quencher” refers to a compound that reduces the emission of a fluorophore when in its presence. Quencher (Q) is selected from a discrete compound, a reactive intermediate which is convertible to a second discrete compound or to a polymerizable compound or Q is a pendant group or chain unit in a polymer prepared from said reactive intermediate or polymerizable compound, which polymer is water-soluble or dispersible or is an insoluble polymer, said polymer is optionally crosslinked.

In certain embodiments, at least one quencher precursor is used to attach the quenching moiety to at least one polymer. For example, aromatic groups may be used to functionalize a viologen with combinations of boronic acid groups and reactive groups. In certain embodiments, this process includes attaching an aromatic group to each of the two nitrogens in the dipyridyl core of the viologen. At least one boronic acid group, a reactive group, or a combination of the two are then attached to each aromatic group, such that the groups attached to each of the two nitrogens on the dipyridyl core of the viologen may either be the same or different. Certain combinations of the functionalized viologen quenching moiety are described as follows:

a) a first aromatic group having a pendent reactive group is attached to the first nitrogen and a second aromatic group having at least one pendent boronic group is attached to the second nitrogen;

b) one or more boronic acid groups are attached to a first aromatic group, which is attached to the first nitrogen, and one boronic acid group and a reactive group are attached to a second aromatic group, which second aromatic group is attached to the second nitrogen;

c) one boronic acid group and a reactive group are attached to a first aromatic group, which first aromatic group is attached to the first nitrogen, and one boronic acid group and a reactive group are attached to a second aromatic group, which is attached to the second nitrogen; and

d) one boronic acid group is attached to an aromatic group, which aromatic group is attached to each of the two nitrogens, and a reactive group is attached to a carbon in a heteroaromatic ring in the heteroaromatic centrally located group.

Preferred embodiments comprise two boronic acid moieties and one polymerizable group or coupling group wherein the aromatic group is a benzyl substituent bonded to the nitrogen and the boronic acid groups are attached to the benzyl ring and may be in the ortho- meta- or para-positions.

In one preferred embodiment, the quencher precursor (before incorporation into a hydrogel) may be 3,3′-oBBV (structure illustrated below); see U.S. Pat. No. 7,470,420, incorporated in its entirety herein by reference thereto.

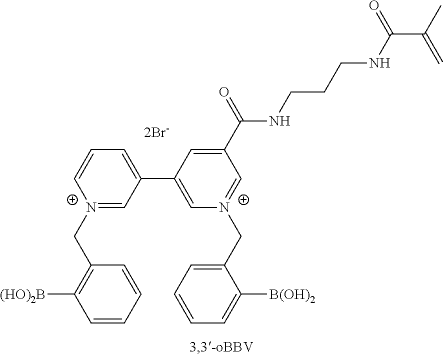

The quencher precursor 3,3′-oBBV may be used with HPTS-CysMA to make hydrogels in accordance with preferred aspects of the invention.

Other indicator chemistries, such as those disclosed in U.S. Pat. Nos. 5,176,882 to Gray et al. and 5,137,833 to Russell, can also be used in accordance with embodiments of the present invention; both of which are incorporated herein in their entireties by reference thereto. In some embodiments, an indicator system may comprise an analyte binding protein operably coupled to a fluorophore, such as the indicator systems and glucose binding proteins disclosed in U.S. Pat. Nos. 6,197,534, 6,227,627, 6,521,447, 6,855,556, 7,064,103, 7,316,909, 7,326,538, 7,345,160, and 7,496,392, U.S. Patent Application Publication Nos. 2003/0232383, 2005/0059097, 2005/0282225, 2009/0104714, 2008/0311675, 2008/0261255, 2007/0136825, 2007/0207498, and 2009/0048430, and PCT International Publication Nos. WO 2009/021052, WO 2009/036070, WO 2009/021026, WO 2009/021039, WO 2003/060464, and WO 2008/072338 which are hereby incorporated by reference herein in their entireties.

For in vivo applications, the sensor is used in a moving stream of physiological fluid which contains one or more polyhydroxyl organic compounds or is implanted in tissue such as muscle which contains said compounds. Therefore, it is preferred that none of the sensing moieties escape from the sensor assembly. Thus, for use in vivo, the sensing components are preferably part of an organic polymer sensing assembly. Soluble dyes and quenchers can be confined by a selectively permeable membrane that allows passage of the analyte but blocks passage of the sensing moieties. This can be realized by using as sensing moieties soluble molecules that are substantially larger than the analyte molecules (molecular weight of at least twice that of the analyte or greater than 1000 preferably greater than 5000); and employing a selective semipermeable membrane such as a dialysis or an ultrafiltration membrane with a specific molecular weight cutoff between the two so that the sensing moieties are quantitatively retained.

Preferably the sensing moieties are immobilized in an insoluble polymer matrix, which is freely permeable to glucose. The polymer matrix is comprised of organic, inorganic or combinations of polymers thereof. The matrix may be composed of biocompatible materials. Alternatively, the matrix is coated with a second biocompatible polymer that is permeable to the analytes of interest.

Although the foregoing invention has been described in terms of certain embodiments and examples, other embodiments will be apparent to those of ordinary skill in the art from the disclosure herein. Moreover, the described embodiments have been presented by way of example only, and are not intended to limit the scope of the inventions. Indeed, the novel methods and systems described herein may be embodied in a variety of other forms without departing from the spirit thereof. Accordingly, other combinations, omissions, substitutions and modifications will be apparent to the skilled artisan in view of the disclosure herein. Thus, the present invention is not intended to be limited by the example or preferred embodiments. The accompanying claims provide exemplary claims and their equivalents are intended to cover forms or modifications as would fall within the scope and spirit of the inventions.

What is claimed is:

1. An analyte detection system, comprising:
an optical fiber sensor comprising a proximal end portion and a distal end portion, wherein a chemical indicator system is disposed along the distal end portion, the chemical indicator system comprising a fluorophore and an analyte binding moiety, which interact to generate a fluorescent signal related to an amount of analyte bound to the analyte binding moiety; and
a deployment device comprising
telescoping tubes configured to extend and retract at least the distal end portion of the optical fiber sensor, such that sensor integrity, hydration and sterility is maintained during sensor calibration and deployment;
a latch disposed on the telescoping tubes and configured to engage with a base disposed on the telescoping tubes, such that when the latch is engaged with the base the distal end portion of the optical fiber sensor extends by a predetermined length; and
an electrical/optical connector disposed at a proximal end of the deployment device and configured to electrically and optically connect with a cable configured to transmit electrical signals and optical signals to and from a monitor apparatus;
wherein the optical fiber sensor is housed concentrically in the telescoping tubes.

2. The analyte detection system of claim 1, wherein the base comprises a gasket configured to create a static seal between the optical fiber sensor and the gasket when the latch and base are engaged.

3. The analyte detection system of claim 2, wherein the gasket is configured to maintain a dynamic seal between the gasket and optical fiber sensor when the latch and base are engaged or opened, wherein the static seal is stronger than the dynamic seal.

4. The analyte detection system of claim 1, further comprising a heating element configured to heat a portion of at least one telescoping tube, wherein the portion of the at least one telescoping tube is configured to house a calibration buffer to calibrate the chemical indicator system.

5. The analyte detection system of claim 4, wherein the heating element comprises a heating tape.

6. The analyte detection system of claim 1, wherein the cable comprises electrical wires configured to transmit the electrical signal and optic fibers configured to transmit the optical signals, wherein the optic signals comprises:
a first plurality of optic fibers configured to transmit a first excitation signal from a first emitter;
a second plurality of optic fibers configured to transmit a second excitation signal from a second emitter;
a third plurality of optic fibers configured to transmit a first fluorescent signal from the chemical indicator system; and
a fourth plurality of optic fibers configured to transmit a second fluorescent signal from the chemical indicator signal.

7. The analyte detection system of claim 6, wherein
the first plurality of optic fibers comprises about two fibers;
the second plurality of optic fibers comprises about three fibers;
the third plurality of optic fibers comprises about twelve fibers; and
the fourth plurality of optic fibers comprises about two fibers.

8. The analyte detection system of claim 1, wherein the electrical/optical connector comprises a housing configured to couple with a monitor connector such that the optical signals between the electrical/optical connector and the cable are stable, the housing comprising one or more optical ferrule coupled with a split sleeve and with a ferrule capture element.

9. The analyte detection system of claim 1, wherein the base further comprises a lock and a key configured to open and close the lock, wherein the key is detachable from the base.

10. The analyte detection system of claim 1, wherein the base comprises a clip, wherein the base position is adjustable along the telescoping tube, wherein the clip comprises a button to allow or disallow an adjustment in the base position.

11. A method of detecting an analyte concentration in a blood vessel, comprising:
introducing a cannula into the blood vessel; connecting a distal end of a deployment device to the cannula, wherein the deployment device comprises telescoping tubes configured to extend and retract at least a distal end portion of an analyte sensor;
deploying the analyte sensor through the cannula and into the blood vessel by sliding the telescoping tubes with respect to one another; and
measuring the analyte concentration by detecting a signal related to the analyte concentration.

12. The method according to claim 11, further comprising:
connecting a calibration buffer injector to the distal end of the deployment device;
injecting a calibration buffer into the deployment device, wherein the distal end portion of the analyte sensor is submerged in the calibration buffer;
heating the calibration buffer to a predetermined temperature with a heating element;
calibrating the analyte sensor; and
removing the calibration buffer from the deployment device.

13. The method according to claim 11, further comprising:
connecting the distal end of the deployment device to a calibration vessel;
extending the distal end portion of the analyte sensor into the calibration vessel by sliding the telescoping tubes with respect to one another;
heating the calibration buffer to a predetermined temperature with a heating element;
calibrating the analyte sensor; and
retracting the distal end portion of the analyte sensor back into the deployment device by sliding the telescoping tubes with respect to one another.

14. The method according to claim 12 or 13, wherein the heating element comprises a heating tape.

15. The method according to claim 11, wherein prior to connecting the distal end of the deployment device to the cannula, the method further comprises:
calibrating the analyte sensor at a predetermined calibration temperature.

16. The method according to claim 11, wherein the deployment device further comprises a catch assembly disposed along the telescoping tubes, the catch assembly being configured to lock the telescoping tubes in a preset position relative to one another, wherein the analyte sensor is extended beyond the distal end of the deployment device when the catch assembly is locked, and wherein the step of deploying the analyte sensor further comprises sliding the telescoping tubes with respect to one another until the catch assembly is locked.

17. The method according to claim 11, further comprising flushing the analyte sensor with a flushing solution.

18. The method according to claim 11, further comprising delivering therapeutic agents.

19. An analyte sensor deployment kit, comprising:
an analyte sensor with proximal and distal end regions, comprising:
an optical fiber with proximal and distal ends;
a chemical indicator system, optically coupled to the optical fiber and disposed along the distal end region of the analyte sensor, the chemical indicator system being capable of generating an emission light signal related to the analyte concentration upon interrogation with an excitation light signal;
a thermocouple disposed along the distal end region of the analyte sensor and comprising a wire that extends proximally along the optical fiber; and
a sensor connector located at the proximal end region of the analyte sensor and coupled to the proximal end of the optical fiber and the thermocouple wire; and
a deployment system, comprising:
a first hollow tube comprising a distal end and a proximal end;
a second hollow tube, slidably and coaxially engaged with the first hollow tube, the second hollow tube comprising a distal end and a proximal end;
a third hollow tube, slidably and coaxially engaged with the second hollow tube, the third hollow tube comprising a distal end and a proximal end;
a connector located at the distal end of the third hollow tube and configured to connect to a first external device; and
a multi-connector junction located at the proximal end of the third hollow tube, configured connect to a second external device;
wherein the analyte sensor is slidably and coaxially engaged within the first, second and third hollow tubes of the deployment system.

20. The analyte sensor deployment kit of claim 19, wherein the first external device is selected from the group consisting of a calibration chamber, a storage chamber, and a cannula.

21. The analyte sensor deployment kit of claim 19, wherein the first external device comprises a calibration chamber comprising a heating element coupled to a monitor configured to control the heating element.

22. The analyte sensor deployment kit of claim 19, wherein the second external device comprises a fluid delivery device.

23. The analyte sensor deployment kit of claim 19, wherein:
the second external device comprises a pressure sensor configured to detect the pressure inside the third tube and to generate a value indicative of a pressure level; and
a pressure control module configured to adjust the pressure inside the third tube depending on the pressure level relative to a predetermined threshold.

24. The analyte sensor deployment kit of claim 19, wherein the second external device comprises a feedback system, comprising:
a blood extracting module configured to extract blood from the patient;
a blood analyte measuring module;
a comparison module configured to compare the analyte concentration measured by the chemical indicator system of the analyte sensor to an analyte concentration measured by the blood analyte measuring module.

25. The analyte sensor deployment kit of claim 19, further comprising a memory device configured to store operational information.
